(19) United States
(12) Patent Application Publication
(10) Pub. No.: US 2023/0265099 A1
(43) Pub. Date: Aug. 24, 2023

(54) SIK INHIBITORS AND METHODS OF USE THEREOF

(71) Applicant: SOLTEGO, INC., Newport Beach, CA (US)

(72) Inventors: Braham SHROOT, Antibes (FR); Beatrice LE VARLET, Montelier (FR)

(73) Assignee: SOLTEGO, INC., Newport Beach, CA (US)

(21) Appl. No.: 18/027,556

(22) PCT Filed: Sep. 21, 2021
(86) PCT No.: PCT/US2021/051375

§ 371 Date: Mar. 21, 2023

Related U.S. Application Data
(60) Provisional application No. 63/081,089, filed on Sep. 21, 2020.

(51) Int. Cl. C07D 487/04; A61P 17/00

(52) U.S. Cl. CPC C07D 487/04; A61P 17/00

(57) ABSTRACT

In one aspect, compounds are provided that can inhibit salt-inducible kinases (SIK) and methods of treating a disease or disorder using the compounds.

### BACKGROUND

Treatments for many dermatological disorders have recognized shortcomings including various side effects and limited effectiveness. Agents that increase skin pigmentation could have many beneficial dermatological effects ranging from improving inflammatory skin disorders and providing sun protection to purely cosmetic applications. It thus would be desirable to have new treatments for dermatological disorders and imperfections.

### CROSS-REFERENCE TO RELATED APPLICATIONS

This application relates to and claims priority from U.S. Patent Application No. 63/081,089 filed on Sep. 21, 2020, the entire disclosure of which is incorporated herein by reference.

### SUMMARY

In one aspect, we provide compounds that inhibit salt-inducible kinases for treatment or prevention of dermatological disorders and skin-related disorders in a subject.

In a preferred aspect, compounds are provided of the following Formula (I):

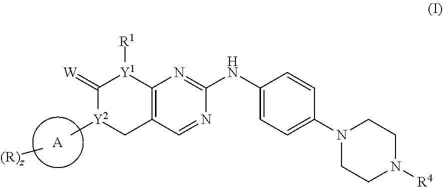

wherein W is S or O;

Y¹ is N or CR^{A};

Y² is N or CR^{B};

Ring A is mono- or multi-ring carbon alicyclic group, mono- or multi-ring heteroalicyclic group, mono- or multi-ring carbocyclic aryl group or mono- or multi-ring heteroaryl group;

each R is the same or different substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine;

z is an integer from 0 (where Ring A has no non-hydrogen ring substituents) to an integer value permitted by the valence of Ring A;

R^{A} and R^{B} are independently H, halogen, —OH, —NH₂, —CN, or substituted or unsubstituted alkyl;

R¹ is substituted or unsubstituted mono- or multi-ring carbon alicyclic group, substituted or unsubstituted mono- or multi-ring heteroalicyclic group, substituted or unsubstituted mono- or multi-ring carbocyclic aryl group or substituted or unsubstituted mono- or multi-ring heteroaryl group; and

R⁴ is H or substituted or unsubstituted alkyl;

and pharmaceutically acceptable salts thereof.

In certain embodiments, Ring A is mono- or multi-ring carbocyclic aryl or multi-ring heteroaryl group. For example, Ring A may be optionally substituted phenyl, optionally substituted naphthyl, or optionally substituted anthracenyl, optionally substituted pyridyl, optionally substituted pyrimidyl, or optionally substituted purinyl. In certain embodiments, Ring A is phenyl, naphthyl, or anthracenyl and z is an integer from 0 to 10. In certain preferred embodiments, Ring A is optionally substituted phenyl.

In certain preferred embodiments, Ring A is a mono- or multi-ring carbon alicyclic group or mono- or multi-ring heteroalicyclic group. In certain preferred embodiments, Ring A is a multi-ring carbon alicyclic group or multi-ring heteroalicyclic group. In certain preferred embodiments, Ring A is a multi-ring carbon alicyclic group.

In certain preferred embodiments, R¹ is a mono- or multi-ring carbon alicyclic group or mono- or multi-ring heteroalicyclic group. In certain preferred embodiments, R¹ is a multi-ring carbon alicyclic group or multi-ring heteroalicyclic group. In certain preferred embodiments, R¹ is a multi-ring carbon alicyclic group.

In certain embodiments, R¹ is mono- or multi-ring carbocyclic aryl or multi-ring heteroaryl group. For example, R¹ may be optionally substituted phenyl, optionally substituted naphthyl, or optionally substituted anthracenyl, optionally substituted pyridyl, optionally substituted pyrimidyl, or optionally substituted purinyl. In certain aspects, R¹ is not a mono-ring group such as phenyl or other mono-carbocyclic aryl or a mono-ring carbon alicyclic.

In certain embodiments of Formula (I), z is an integer of 0 to 20, more typically z is 0, 1, 2, 3, 4, 5, 6, 7, 8, 9 or 10, or in certain embodiments z is 0, 1, 2, 3, 4, 5 or 6, or in certain embodiments z is 0, 1, 2 or 3.

In certain embodiments of Formula I, R is substituted or unsubstituted alkyl or halogen such as F, Cl, Br or I, and particularly Cl. In certain embodiments of Formula I, R is substituted or unsubstituted alkoxy such as methoxy or ethoxy.

In certain aspects, one or more R groups is substituted or unsubstituted alkyl and does not contain any unsaturated carbon-carbon bonds. In certain aspects, one or more R groups is substituted alkyl such as C₁₋₆ unsubstituted alkyl and does not contain any unsaturated carbon-carbon bonds. In other aspects, R is substituted or unsubstituted alkyl and may contain one or more unsaturated carbon-carbon bonds (and thus such R may be referred to as substituted or unsubstituted alkenyl such as having 2 to 6 or 8 carbon atoms or substituted or u unsubstituted alkynyl such as having 2 to 6 or 8 carbon atoms).

In preferred aspect, compounds are provided of the following Formula (II):

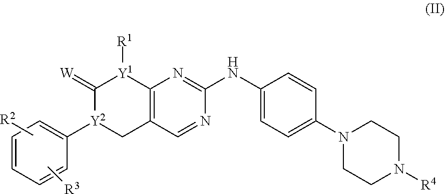

wherein W is S or O;

Y¹ is N or CR^{A};

Y² is N or CR^{B};

R^{A} and R^{B} are independently H, halogen, —OH, —NH₂, —CN, or substituted or unsubstituted alky;

R¹ is substituted or unsubstituted multi-ring carbon alicyclic group or substituted or unsubstituted multi-ring heteroalicyclic group;

R² and R³ are each independently H, substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine; and

R⁴ is H or substituted or unsubstituted alkyl;

and pharmaceutically acceptable salts thereof.

Preferred compounds of Formula (I) or (II) include those of the following Formula (III):

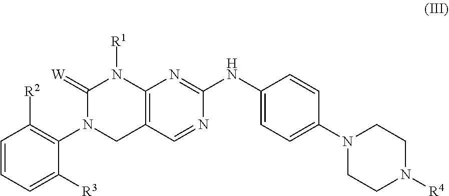

wherein in Formula (III), W, R¹, R², R³, and R⁴ are each the same as defined above for Formula (II); and pharmaceutically acceptable salts thereof.

In a further aspect, compounds of the following Formula (IV) are provided:

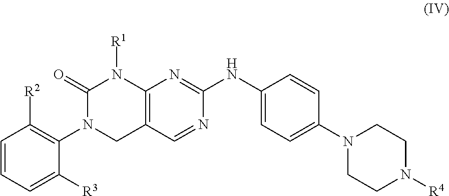

wherein in Formula (IV) R¹, R², R³, and R⁴ are each the same as defined above for Formula (II); and pharmaceutically acceptable salts thereof.

In certain embodiments, in any of Formulae (II), (III) or (IV), each of R² and R³ is independently H, substituted or unsubstituted C₁-C₆ alkyl, or halogen, and R⁴ is H or substituted or unsubstituted C₁-C₆ alkyl. In certain embodiments, at least one of R² and R³ is substituted or unsubstituted C₁-C₄ alkyl, such as optionally substituted methyl, or halogen such as —F, —Cl, —Br, or —I. In certain embodiments of Formulae (II), (III) or (IV), both of R² and R³ are other than H, and may be in certain preferred aspects substituted or unsubstituted C₁-C₄ alkyl, such as optionally substituted methyl, or halogen such as —F, —Cl, —Br or —I, particularly —Cl.

In particular embodiments, in any of Formula (II), (III) or (IV), R², R³, and R⁴ are the same or different and are each unsubstituted C₁-C₄ alkyl. In certain embodiments, one or more of R², R³, and R⁴ is unsubstituted methyl. In certain embodiments, at least two or more of R², R³ and R⁴ is unsubstituted methyl. In certain embodiments, each of R², R³, and R⁴ is unsubstituted methyl.

In particular embodiments, in any of Formula (II), (III) or (IV), R² and R³ are each independently unsubstituted C₁-C₄ alkyl or halogen. In certain embodiments, at least one of R² and R³ is unsubstituted methyl. In certain embodiments, at least one of R² and R³ is halogen. In certain embodiments, at least one of R² and R³ is —Cl. In certain embodiments, at least one of R² and R³ is —Br. In certain embodiments, at least one of R² and R³ is —F. In certain embodiments, at least one of R² and R³ is —I.

In certain embodiments, preferred compounds include those that have a structure of the following Formula (V):

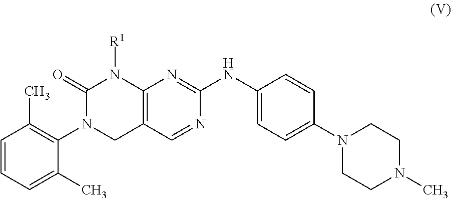

wherein in Formula (V), R¹ is the same as defined in Formulae (I) or (II) above; and pharmaceutically acceptable salts thereof.

In particular aspects, preferred compounds of the above Formula (I), (II), (III), (IV), or (V) include those where R¹ is an optionally substituted multi-ring carbon alicyclic group.

In a preferred embodiment of any one of the above formulae, R¹ comprises a bicyclic carbon alicyclic group. In another preferred embodiment, R¹ comprises a tricyclic carbon alicyclic group.

More particularly, in one aspect, preferred are compounds of Formula (VI) which are defined as compounds of any of the above Formulae (I), (II), (III), (IV) and/or (V) where R¹ has structure of the following Formula (VIa):

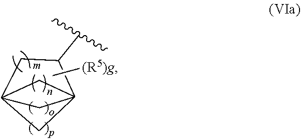

wherein in Formula (VIa):

m is an integer from 0 to 5;

n is an integer from 0 to 3;

o is an integer from 0 to 3;

p is an integer from 0 to 5,

each R⁵ is the same or different non-hydrogen substituent such as hydroxyl, halo, optionally substituted alkyl or optionally substituted heteroalkyl,

g is 0 (where no R⁵ groups are present) or a positive integer such as from 1 to 10, and more typically g is 0, 1, 2, 3, 4, 5, or 6. In certain embodiments of Formula (VIa), and at least one of n and o is a positive integer, when n or o is 0, there is no direct bond formed between the two adjacent carbon atoms, and when m or p is 0, there is a direct bond formed between the two adjacent carbon atoms. In certain aspects, g will be 0, i.e. the carbon alicyclic ring will not have any non-hydrogen substituents other than a linkage to the ring nitrogen (it being recognized that the compound ring nitrogen not shown in Formula (VIa) above but the linkage of R¹ to the ring nitrogen of a compound of Formula (I), (II), (III), (IV), (V) or (VI) designated by the wavy line of Formula VIa). In certain aspects, g is 0, 1, 2 or 3, or g is 0, 1 or 2.

In certain embodiments of Formulae (VI) and (VIa), at least one of n and o is an integer of 1 or greater.

In additional certain embodiments of the above formulae including Formulae (VI) and (VIa), R¹ may be optionally substituted adamantyl.

In still further embodiments of the above formulae including Formulae (VI) and (VIa), R¹ may be optionally substituted norbornyl.

In yet further embodiments of the above formulae including Formulae (VI) and (VIa), R¹ may be optionally substituted bicyclo[2,2,2] octanyl.

In additional embodiments of the above formulae including Formulae (VI) and (VIa), R¹ may be optionally substituted bicyclo[3,3,1] nonanyl.

In related embodiments, in any of Formulae (I), (II), (III), (IV), (V) and/or (VI), R¹ may include a moiety selected from the following:

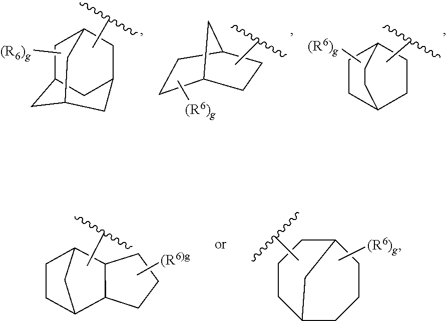

wherein in those structures each R⁶ may be the same or different non-hydrogen substituent such as hydroxyl, halo, optionally substituted C₁₋₆ alkyl or optionally substituted C₁₋₆ heteroalkyl such as optionally substituted C₁₋₆ alkoxy, and g may be 0 (where no R⁶ groups are present) or a positive integer such as from 1 to 10, or more typically 1, 2, 3, 4, 5, or 6. In certain embodiments, g will be zero, i.e. the depicted carbon alicyclic rings will not have any non-hydrogen substituents other than a linkage to the ring nitrogen. As will be understood, the wavy line in those above structures designates a linkage to the ring nitrogen of a compound of Formulae (I), (II), (III), (IV), (V) and/or (VI).

In certain preferred embodiments, g is 0 in the above structures. In such embodiments, in compounds of Formulae (I), (II), (III), (IV), (V) and/or (VI) R¹ may comprise a moiety selected from the following:

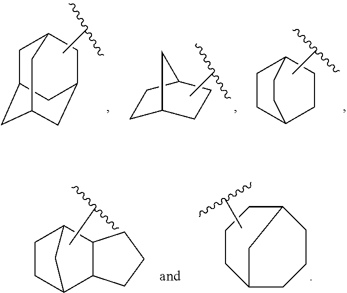

Again, as will be understood, the wavy line in those above structures designates a linkage to the ring nitrogen of a compound of Formulae (I), (II), (III), (IV), (V) and/or (VI).

In additional embodiments, preferred compounds include of any of the following structures:

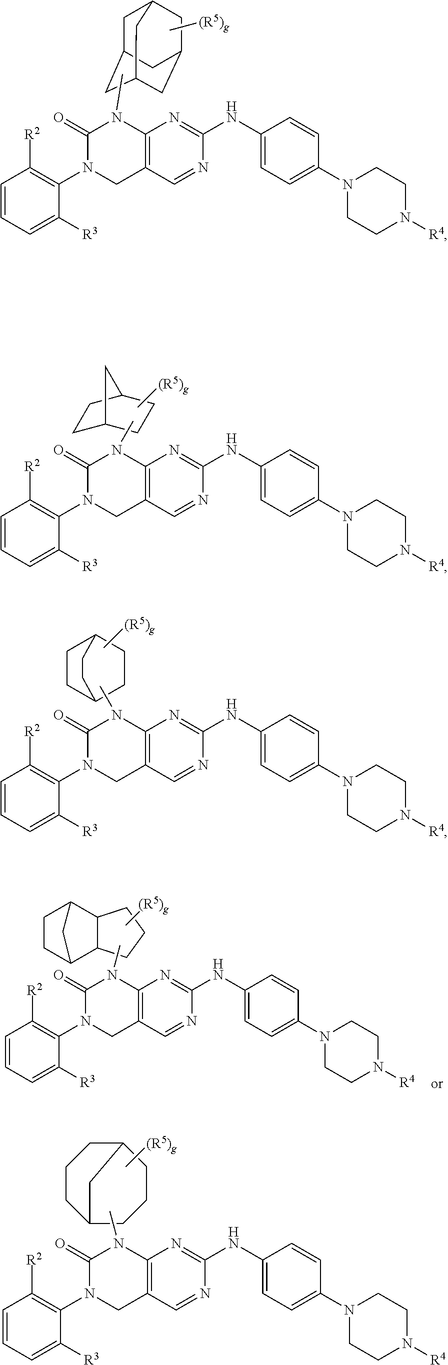

wherein in the above structures R², R³, and R⁴ are the same as defined in Formula (II) above; and R⁵ and g are the same as defined in Formula (VIa) above; and pharmaceutically acceptable salts thereof. R², R³, R⁴, and each R⁵ preferably may be optionally substituted C₁₋₄ alkyl such as optionally substituted methyl. Preferably, g may be 0, 1, 2 or 3.

In additional embodiments, preferred compounds include of any of the following structures:

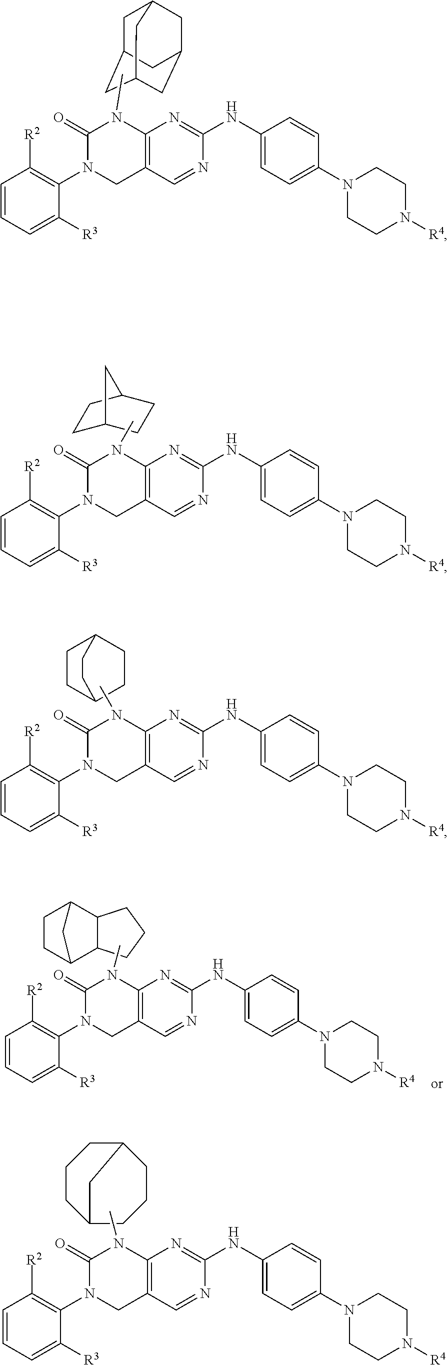

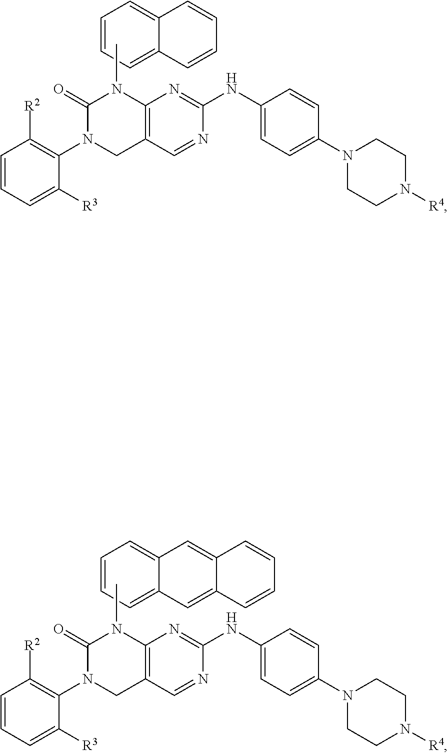

wherein in the above structures R², R³, and R⁴ are the same as defined in Formulae (II) above; and pharmaceutically acceptable salts thereof. R², R³, and R⁴ preferably may be optionally substituted C₁₋₄ alkyl such as optionally substituted methyl including unsubstituted methyl. In certain embodiments, R², R³, and R⁴ are each unsubstituted methyl. In certain embodiments, at least one of R² and R³ is unsubstituted methyl. In certain embodiments, at least one of R² and R³ is halogen.

In additional embodiments, compounds of the above Formulae (I), (II), (III), (IV), (V) and/or (VI) include those where R¹ is an optionally substituted multi-ring heteroalicyclic group. For instance, suitable R¹ groups may include a moiety comprising optionally substituted thionorbonyl or optionally substituted oxonorbonyl.

In a further aspect, pharmaceutical compositions are provided comprising a compound of any one of Formulae (I), (II), (III), (IV), (V) and/or (VI) as set forth above. The compositions suitably may comprise one or more pharmaceutically acceptable carriers. In preferred embodiments, the compositions may be formulated or otherwise adapted for a skin pigmentation-related condition, for instance the composition may be adapted for topical administration such as an ointment, gel or lotion, or for oral administration as a tablet or capsule.

In preferred aspects, methods are provided to increase pigmentation in a tissue of a subject, comprising administering to the subject a compound of any one of Formulae (I), (II), (III), (IV), (V) and/or (VI) as set forth above, in an amount sufficient to increase melanin production, thereby increasing pigmentation in the tissue of the subject.

In other preferred aspects, methods are provided to treat a subject suffering from or susceptible to inflammatory dermatosis, including any of erythematotelangiectatic rosacea (subtype 1 rosacea), papulopustular rosacea (subtype 2 rosacea), phymatous rosacea (subtype 3 rosacea) and/or ocular rosacea (subtype 4 rosacea).

In preferred other aspects, methods are provided to increase cellular DNA stability in the skin tissue of a subject in need thereof, comprising administering to the subject a compound of any one of Formulae (I), (II), (III), (IV), (V) and/or (VI) as set forth above, in an amount sufficient to decrease apoptosis and/or thymine dimer formation in the cellular DNA of the skin tissue, thereby increasing cellular DNA stability in the skin tissue of the subject.

In a yet further aspect, kits are provided for use to treat or prevent a disease or disorder including pigmentation disorders, unevenness of skin tone, hypopigmentation, vitiligo, inflammatory dermatosis, including rosacea, or other skin-related disorders or conditions. Kits of the invention suitably may comprise 1) one or more compounds of any of Formulae (I), (II), (III), (IV), (V) or (VI); and 2) instructions for using the one or more compounds for treating or preventing a disease or disorder including hypopigmentation, vitiligo, inflammatory dermatosis, including rosacea or other skin-related disorders or conditions. Preferably, a kit will comprise a therapeutically effective amount of one or more compounds of any of Formulae (I), (II), (III), (IV), (V) or (VI). The instructions suitably may be in written form, including as a product label.

Methods of treating various skin conditions, such as for cosmetic purposes, can be carried out using the systems described herein. It is understood that although such methods can be conducted by a physician, non-physicians, such as aestheticians and other suitably trained personnel may use the systems described herein to treat various skin conditions with and without the supervision of a physician.

Other aspects of the invention are disclosed infra.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIG. 1 depicts melanin content in Fontana-Masson stained sections of vehicle (PEG/Ethanol/Transcutol®) and SLT-008, 3 mM.

FIG. 2 depicts the effects of SLT-008 on melanin synthesis, compared to the excipient E2-treated stripped batch SE2J11. Black: Control, Red: UV control, Purple: 0.5% SLT-008 and Blue: 0.9% SLT-008.

FIG. 3 depicts melanin content determined from Fontana-Masson stained sections of vehicle (absolute ethanol) and SLT-008 (0.9%).

FIG. 4 depicts tissues having different doses of SLT-008 (0.3 μg/ml, 0.01 μg/ml, and 0.0033 μg/ml) fixed in 4% formaldehyde, dehydrated and paraffin embedded. Sections of 6 μm of epidermis were stained with eosin and hematoxylin (H/E). Slides were mounted with specific medium and examined with a Leica DM2000 photomicroscope coupled to a digital camera (Zeiss).

FIG. 5 depicts lactate dehydrogenase (LDH) release following the application of SLT-008 for 10 days on melanized reconstructed epidermis.

FIG. 6 depicts melanin content in Fontana-Masson stained sections. Epidermal pigmentation is shown by a dendritic morphology of functional melanocytes, and the formation of melanosomes organized as supranuclear melanin caps above the keratinocyte nuclei. FIG. 6A shows the untreated control, the vehicle (DMSO 0.05%) and two positive controls, forskolin (3.33 μM) or IBMX (150 μM) vs DMSO 0.05%. FIG. 6B shows SLT-008 at 0.3 μg/ml, 0.011 μg/ml, 0.0033 μg/ml vs DMSO 0.005%.

FIG. 7 depicts melanin content determined from Fontana-Masson stained sections (% relative to DMSO 0.05% for IBMX and FSK; % relative to DMSO 0.005% for SLT-008 treatments). IBMX dosed at 150 μM and forskolin dosed at 3.33 μM were used as pro-pigmentation controls. The results are as shown. SLT-008 dosed at 0.3 μg/ml scored 129+/−5.7 melanin content. SLT-008 dosed at 0.011 μg/ml scored 125.6+/−15.4 melanin content.

FIG. 8 depicts melanin content by chemical extraction following application of SLT-008 at 1.0 μg/ml, 0.1 μg/ml and 0.3 μg/ml

FIG. 9 depicts the percentage of surface positive for TUNEL staining related to apoptotic cells in the epidermis for all samples.

FIG. 10 depicts the percentage of surface positive for thymine dimers in the epidermis for all samples.

FIG. 11 depicts the percentage of surface positive for TUNEL staining related to apoptotic cells in the epidermis for all samples.

### DETAILED DESCRIPTION

As discussed, we now provide new compounds and methods, including compounds of Formulae (I), (II), (III), (IV), (V) and (VI).

Specifically preferred compounds include the following and pharmaceutically acceptable salts of these compounds:

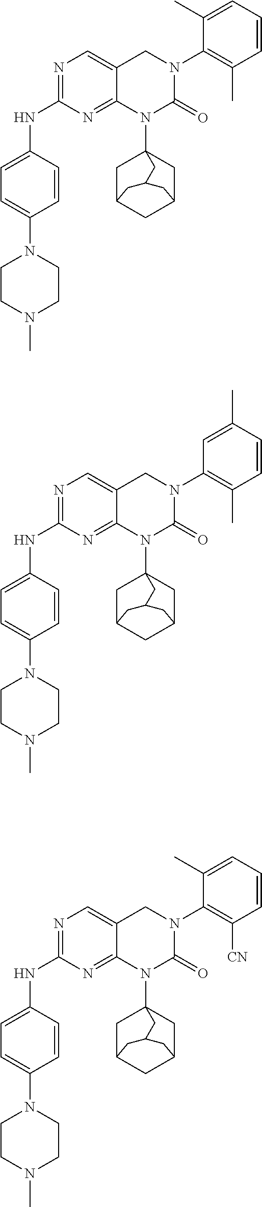 [SMILES: Cc1cccc(C)c1N1Cc2cnc(Nc3ccc(N4CCN(C)CC4)cc3)nc2N(C23CC4CC(CC(C4)C2)C3)C1=O ; Cc1ccc(C)c(N2Cc3cnc(Nc4ccc(N5CCN(C)CC5)cc4)nc3N(C34CC5CC(CC(C5)C3)C4)C2=O)c1 ; Cc1cccc(C#N)c1N1Cc2cnc(Nc3ccc(N4CCN(C)CC4)cc3)nc2N(C23CC4CC(CC(C4)C2)C3)C1=O]

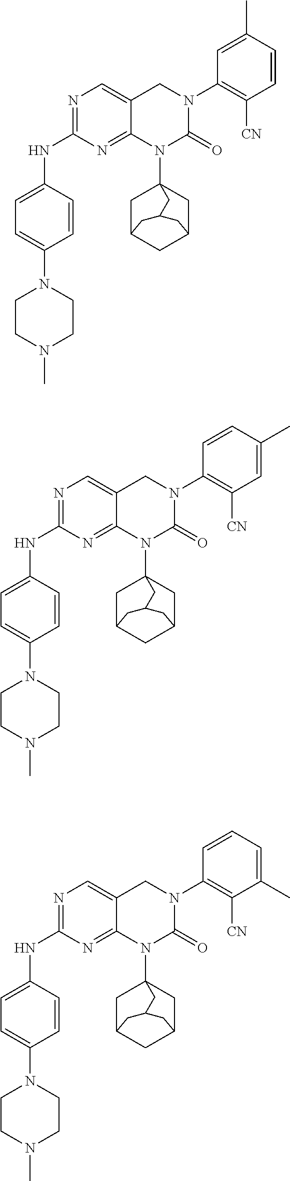 [SMILES: Cc1ccc(C#N)c(N2Cc3cnc(Nc4ccc(N5CCN(C)CC5)cc4)nc3N(C34CC5CC(CC(C5)C3)C4)C2=O)c1 ; Cc1ccc(N2Cc3cnc(Nc4ccc(N5CCN(C)CC5)cc4)nc3N(C34CC5CC(CC(C5)C3)C4)C2=O)c(C#N)c1 ; Cc1cccc(N2Cc3cnc(Nc4ccc(N5CCN(C)CC5)cc4)nc3N(C34CC5CC(CC(C5)C3)C4)C2=O)c1C#N]

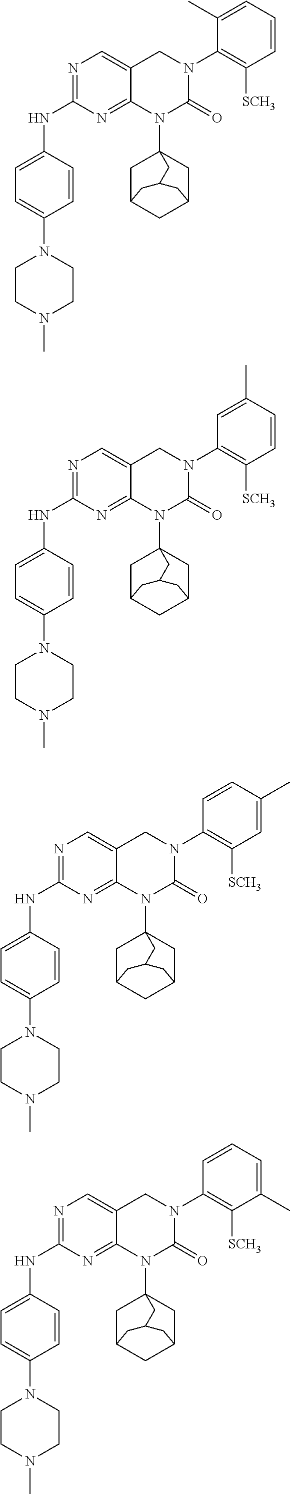 [SMILES: CSc1cccc(C)c1N1Cc2cnc(Nc3ccc(N4CCN(C)CC4)cc3)nc2N(C23CC4CC(CC(C4)C2)C3)C1=O ; CSc1ccc(C)cc1N1Cc2cnc(Nc3ccc(N4CCN(C)CC4)cc3)nc2N(C23CC4CC(CC(C4)C2)C3)C1=O ; CSc1cc(C)ccc1N1Cc2cnc(Nc3ccc(N4CCN(C)CC4)cc3)nc2N(C23CC4CC(CC(C4)C2)C3)C1=O ; CSc1c(C)cccc1N1Cc2cnc(Nc3ccc(N4CCN(C)CC4)cc3)nc2N(C23CC4CC(CC(C4)C2)C3)C1=O]

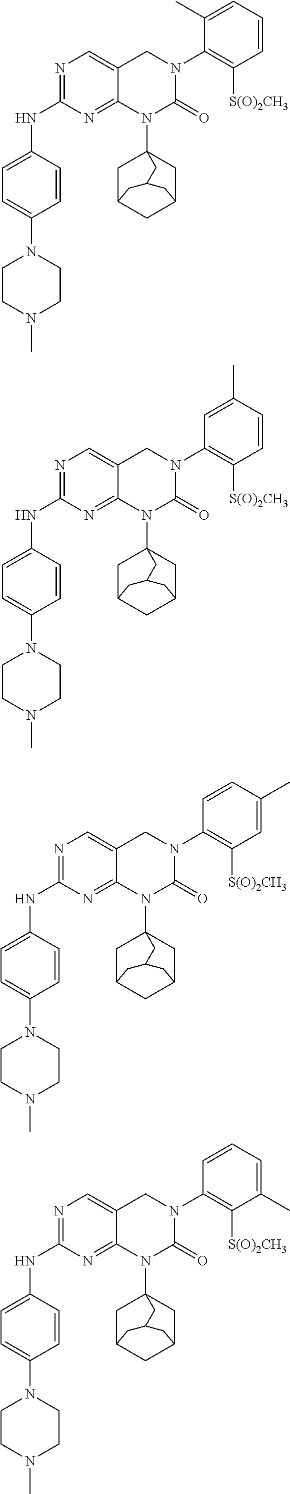

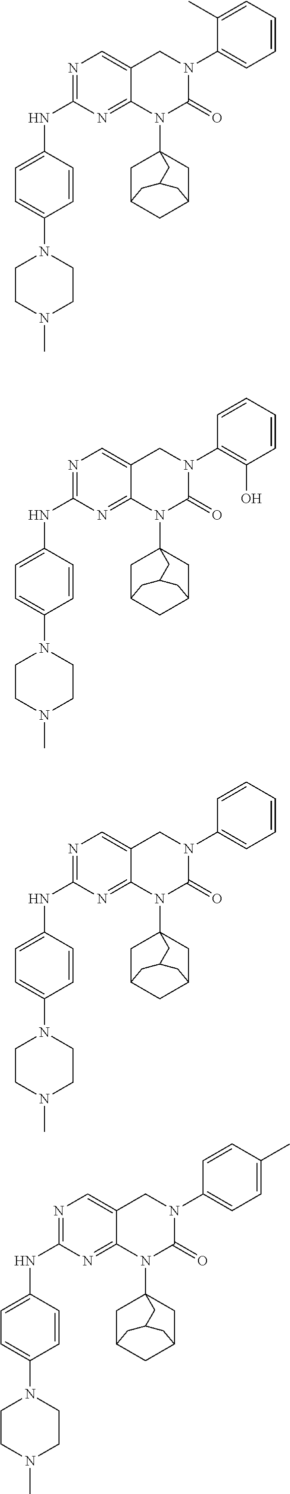 [SMILES: Cc1ccccc1N1Cc2cnc(Nc3ccc(N4CCN(C)CC4)cc3)nc2N(C23CC4CC(CC(C4)C2)C3)C1=O ; CN1CCN(c2ccc(Nc3ncc4c(n3)N(C35CC6CC(CC(C6)C3)C5)C(=O)N(c3ccccc3O)C4)cc2)CC1 ; CN1CCN(c2ccc(Nc3ncc4c(n3)N(C35CC6CC(CC(C6)C3)C5)C(=O)N(c3ccccc3)C4)cc2)CC1 ; Cc1ccc(N2Cc3cnc(Nc4ccc(N5CCN(C)CC5)cc4)nc3N(C34CC5CC(CC(C5)C3)C4)C2=O)cc1]

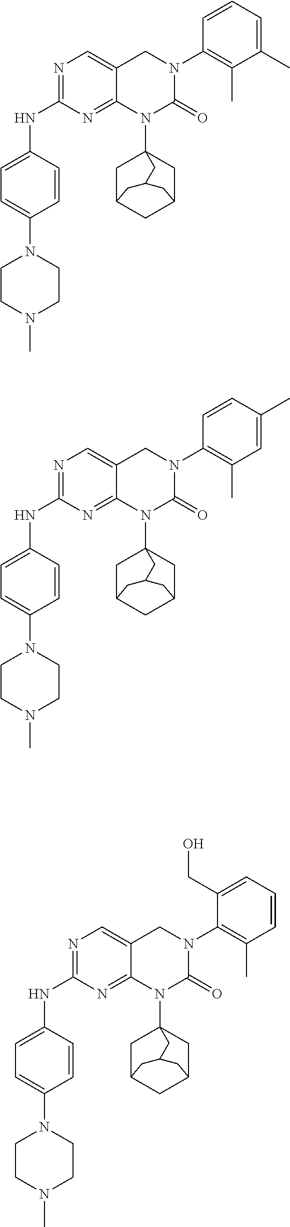 [SMILES: Cc1cccc(N2Cc3cnc(Nc4ccc(N5CCN(C)CC5)cc4)nc3N(C34CC5CC(CC(C5)C3)C4)C2=O)c1C ; Cc1ccc(N2Cc3cnc(Nc4ccc(N5CCN(C)CC5)cc4)nc3N(C34CC5CC(CC(C5)C3)C4)C2=O)c(C)c1 ; Cc1cccc(CO)c1N1Cc2cnc(Nc3ccc(N4CCN(C)CC4)cc3)nc2N(C23CC4CC(CC(C4)C2)C3)C1=O]

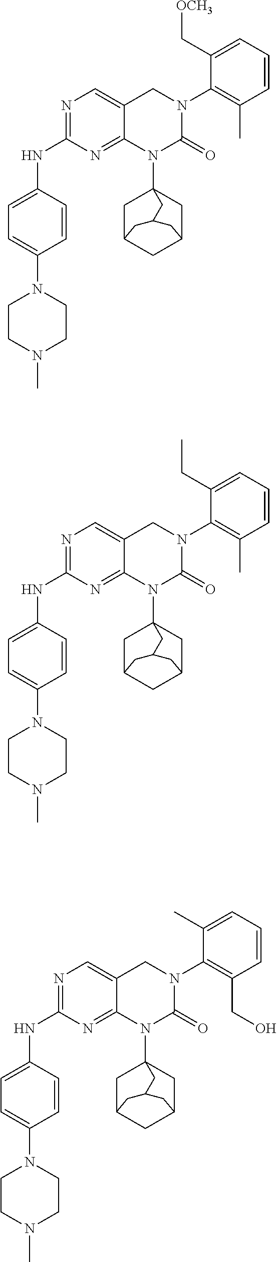 [SMILES: COCc1cccc(C)c1N1Cc2cnc(Nc3ccc(N4CCN(C)CC4)cc3)nc2N(C23CC4CC(CC(C4)C2)C3)C1=O ; CCc1cccc(C)c1N1Cc2cnc(Nc3ccc(N4CCN(C)CC4)cc3)nc2N(C23CC4CC(CC(C4)C2)C3)C1=O ; Cc1cccc(CO)c1N1Cc2cnc(Nc3ccc(N4CCN(C)CC4)cc3)nc2N(C23CC4CC(CC(C4)C2)C3)C1=O]

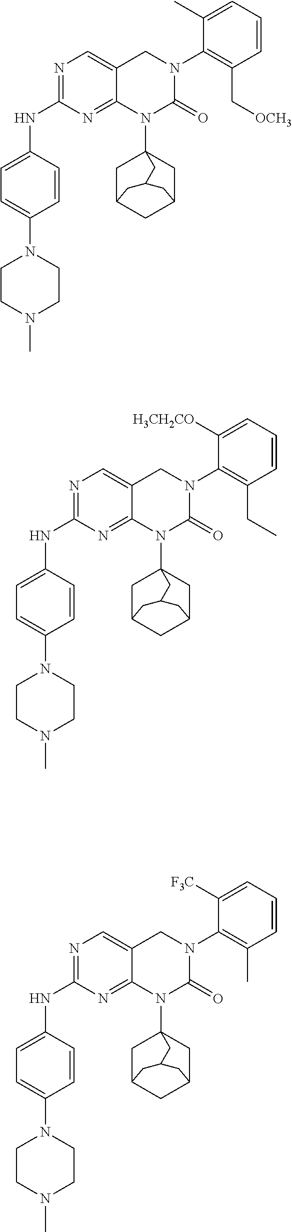

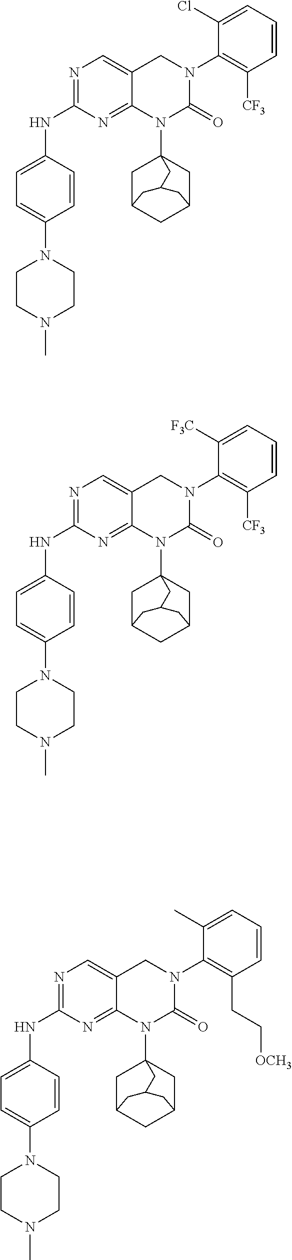 [SMILES: CN1CCN(c2ccc(Nc3ncc4c(n3)N(C35CC6CC(CC(C6)C3)C5)C(=O)N(c3c(Cl)cccc3C(F)(F)F)C4)cc2)CC1 ; CN1CCN(c2ccc(Nc3ncc4c(n3)N(C35CC6CC(CC(C6)C3)C5)C(=O)N(c3c(C(F)(F)F)cccc3C(F)(F)F)C4)cc2)CC1 ; COCCc1cccc(C)c1N1Cc2cnc(Nc3ccc(N4CCN(C)CC4)cc3)nc2N(C23CC4CC(CC(C4)C2)C3)C1=O]

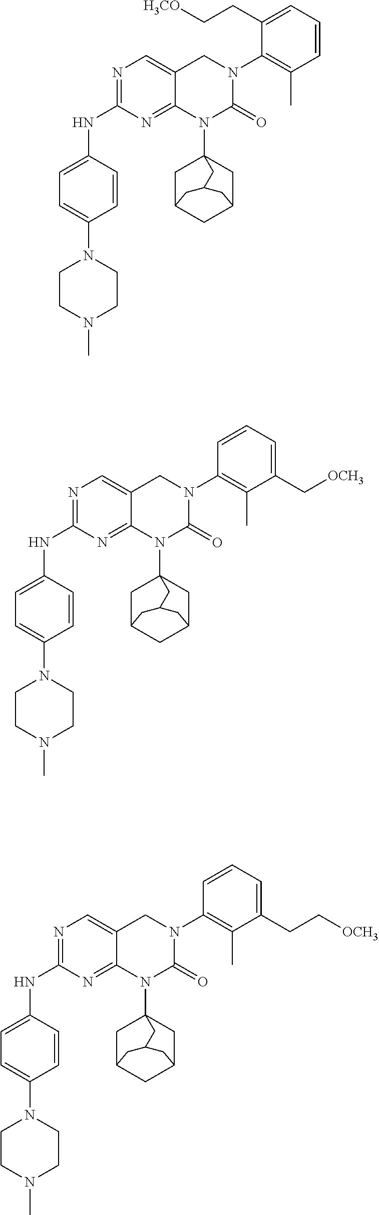 [SMILES: COCCc1cccc(C)c1N1Cc2cnc(Nc3ccc(N4CCN(C)CC4)cc3)nc2N(C23CC4CC(CC(C4)C2)C3)C1=O ; COCc1cccc(N2Cc3cnc(Nc4ccc(N5CCN(C)CC5)cc4)nc3N(C34CC5CC(CC(C5)C3)C4)C2=O)c1C ; COCCc1cccc(N2Cc3cnc(Nc4ccc(N5CCN(C)CC5)cc4)nc3N(C34CC5CC(CC(C5)C3)C4)C2=O)c1C]

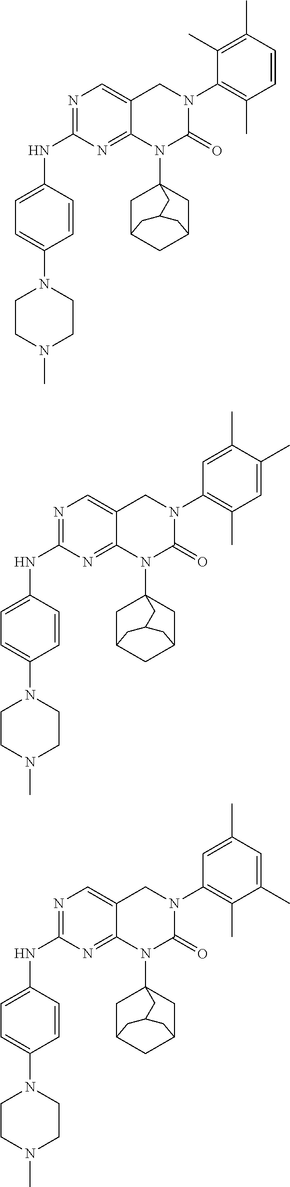 [SMILES: Cc1ccc(C)c(N2Cc3cnc(Nc4ccc(N5CCN(C)CC5)cc4)nc3N(C34CC5CC(CC(C5)C3)C4)C2=O)c1C ; Cc1cc(C)c(N2Cc3cnc(Nc4ccc(N5CCN(C)CC5)cc4)nc3N(C34CC5CC(CC(C5)C3)C4)C2=O)cc1C ; Cc1cc(C)c(C)c(N2Cc3cnc(Nc4ccc(N5CCN(C)CC5)cc4)nc3N(C34CC5CC(CC(C5)C3)C4)C2=O)c1]

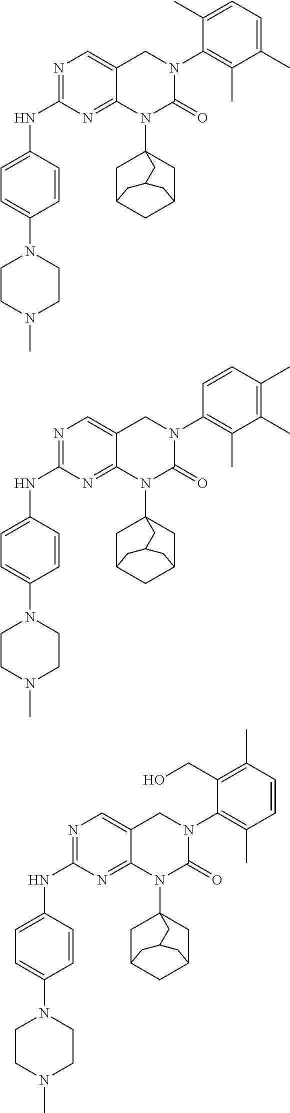 [SMILES: Cc1ccc(C)c(N2Cc3cnc(Nc4ccc(N5CCN(C)CC5)cc4)nc3N(C34CC5CC(CC(C5)C3)C4)C2=O)c1C ; Cc1ccc(N2Cc3cnc(Nc4ccc(N5CCN(C)CC5)cc4)nc3N(C34CC5CC(CC(C5)C3)C4)C2=O)c(C)c1C ; Cc1ccc(C)c(N2Cc3cnc(Nc4ccc(N5CCN(C)CC5)cc4)nc3N(C34CC5CC(CC(C5)C3)C4)C2=O)c1CO]

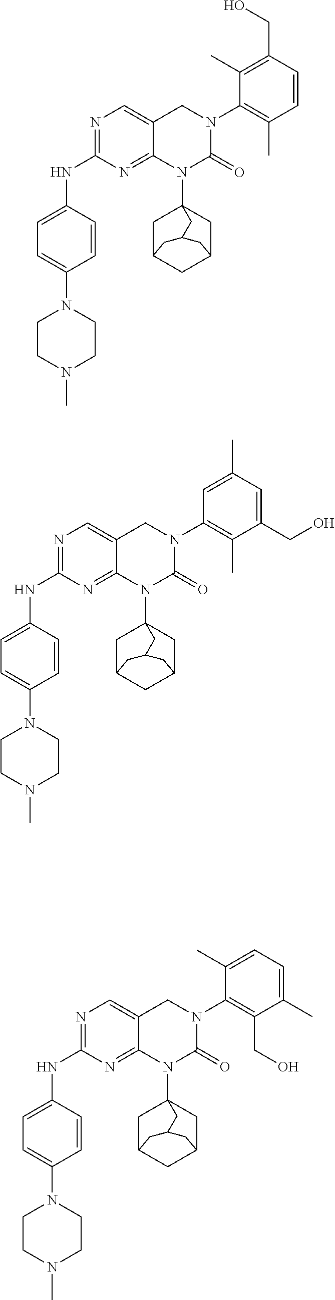 [SMILES: Cc1ccc(CO)c(C)c1N1Cc2cnc(Nc3ccc(N4CCN(C)CC4)cc3)nc2N(C23CC4CC(CC(C4)C2)C3)C1=O ; Cc1cc(CO)c(C)c(N2Cc3cnc(Nc4ccc(N5CCN(C)CC5)cc4)nc3N(C34CC5CC(CC(C5)C3)C4)C2=O)c1 ; Cc1ccc(C)c(N2Cc3cnc(Nc4ccc(N5CCN(C)CC5)cc4)nc3N(C34CC5CC(CC(C5)C3)C4)C2=O)c1CO]

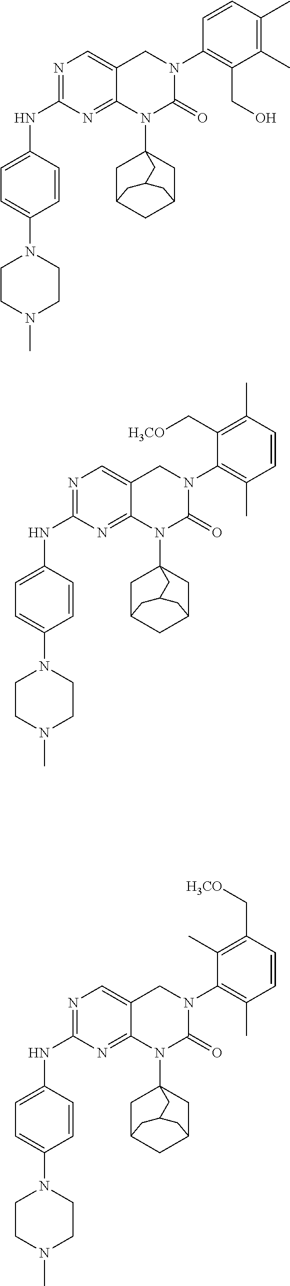 [SMILES: Cc1ccc(N2Cc3cnc(Nc4ccc(N5CCN(C)CC5)cc4)nc3N(C34CC5CC(CC(C5)C3)C4)C2=O)c(CO)c1C ; COCc1c(C)ccc(C)c1N1Cc2cnc(Nc3ccc(N4CCN(C)CC4)cc3)nc2N(C23CC4CC(CC(C4)C2)C3)C1=O ; COCc1ccc(C)c(N2Cc3cnc(Nc4ccc(N5CCN(C)CC5)cc4)nc3N(C34CC5CC(CC(C5)C3)C4)C2=O)c1C]

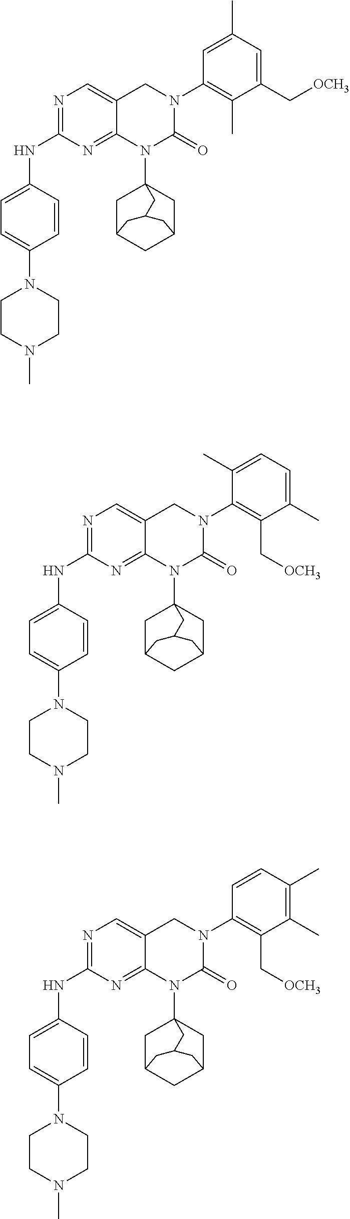 [SMILES: COCc1cc(C)cc(N2Cc3cnc(Nc4ccc(N5CCN(C)CC5)cc4)nc3N(C34CC5CC(CC(C5)C3)C4)C2=O)c1C ; COCc1c(C)ccc(C)c1N1Cc2cnc(Nc3ccc(N4CCN(C)CC4)cc3)nc2N(C23CC4CC(CC(C4)C2)C3)C1=O ; COCc1c(N2Cc3cnc(Nc4ccc(N5CCN(C)CC5)cc4)nc3N(C34CC5CC(CC(C5)C3)C4)C2=O)ccc(C)c1C]

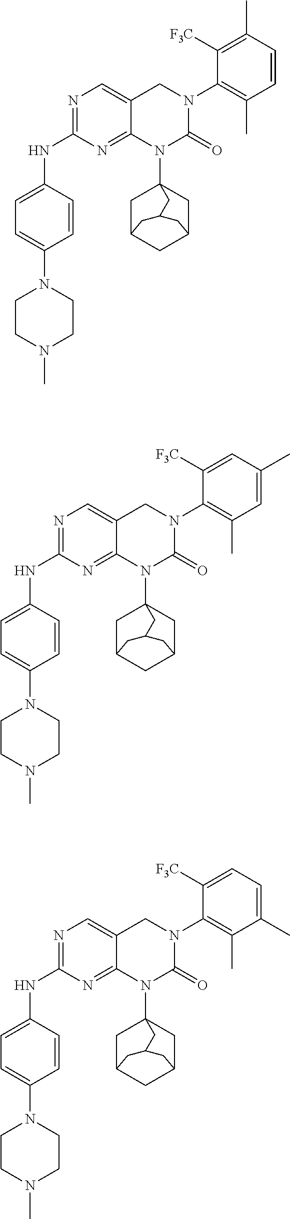 [SMILES: Cc1ccc(C)c(C(F)(F)F)c1N1Cc2cnc(Nc3ccc(N4CCN(C)CC4)cc3)nc2N(C23CC4CC(CC(C4)C2)C3)C1=O ; Cc1cc(C)c(N2Cc3cnc(Nc4ccc(N5CCN(C)CC5)cc4)nc3N(C34CC5CC(CC(C5)C3)C4)C2=O)c(C(F)(F)F)c1 ; Cc1ccc(C(F)(F)F)c(N2Cc3cnc(Nc4ccc(N5CCN(C)CC5)cc4)nc3N(C34CC5CC(CC(C5)C3)C4)C2=O)c1C]

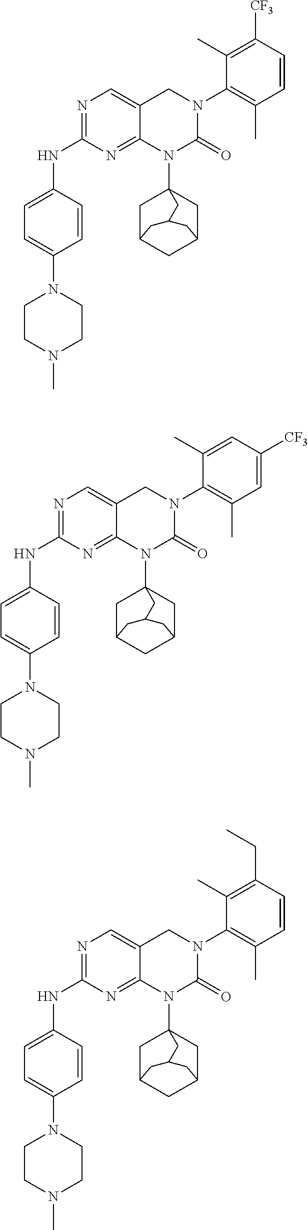 [SMILES: Cc1ccc(C(F)(F)F)c(C)c1N1Cc2cnc(Nc3ccc(N4CCN(C)CC4)cc3)nc2N(C23CC4CC(CC(C4)C2)C3)C1=O ; Cc1cc(C(F)(F)F)cc(C)c1N1Cc2cnc(Nc3ccc(N4CCN(C)CC4)cc3)nc2N(C23CC4CC(CC(C4)C2)C3)C1=O ; CCc1ccc(C)c(N2Cc3cnc(Nc4ccc(N5CCN(C)CC5)cc4)nc3N(C34CC5CC(CC(C5)C3)C4)C2=O)c1C]

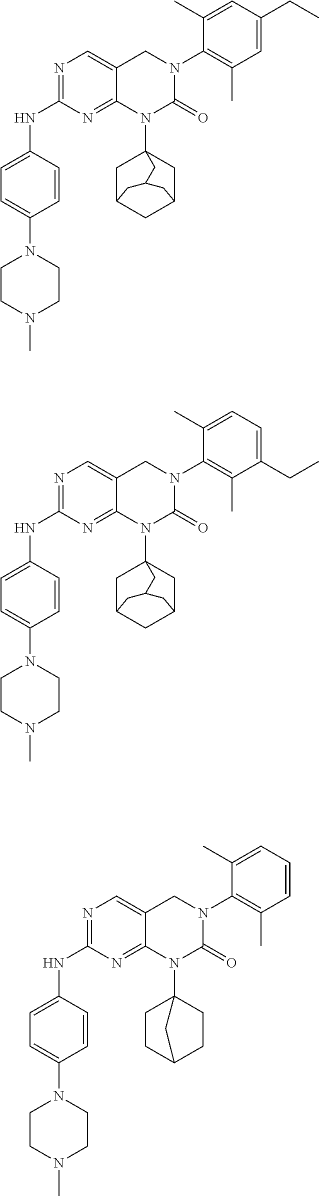 [SMILES: CCc1cc(C)c(N2Cc3cnc(Nc4ccc(N5CCN(C)CC5)cc4)nc3N(C34CC5CC(CC(C5)C3)C4)C2=O)c(C)c1 ; CCc1ccc(C)c(N2Cc3cnc(Nc4ccc(N5CCN(C)CC5)cc4)nc3N(C34CC5CC(CC(C5)C3)C4)C2=O)c1C ; Cc1cccc(C)c1N1Cc2cnc(Nc3ccc(N4CCN(C)CC4)cc3)nc2N(C23CCC(CC2)C3)C1=O]

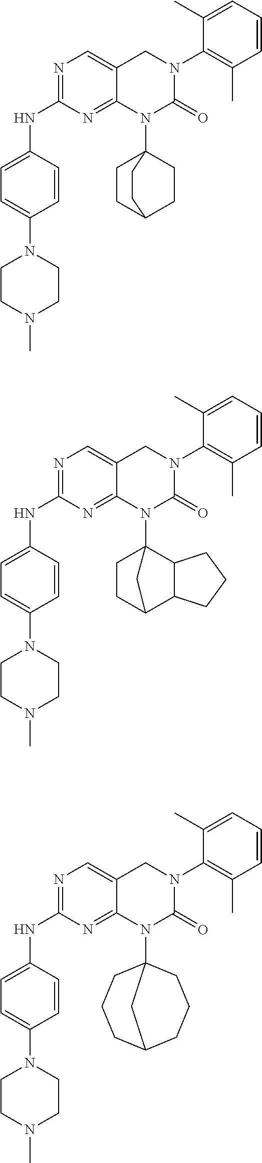 [SMILES: Cc1cccc(C)c1N1Cc2cnc(Nc3ccc(N4CCN(C)CC4)cc3)nc2N(C23CCC(CC2)CC3)C1=O ; Cc1cccc(C)c1N1Cc2cnc(Nc3ccc(N4CCN(C)CC4)cc3)nc2N(C23CCC(C2)C2CCCC23)C1=O ; Cc1cccc(C)c1N1Cc2cnc(Nc3ccc(N4CCN(C)CC4)cc3)nc2N(C23CCCC(CCC2)C3)C1=O]

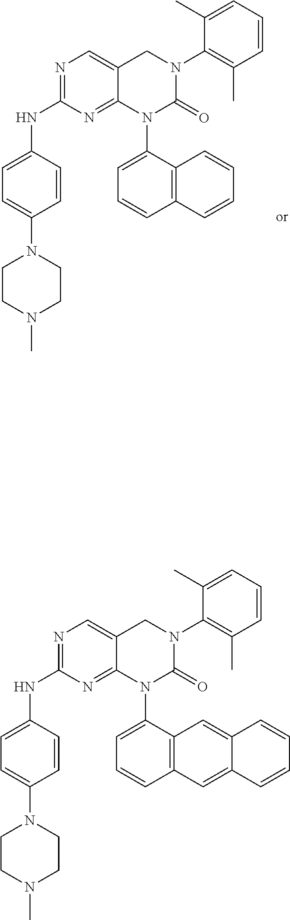 [SMILES: Cc1cccc(C)c1N1Cc2cnc(Nc3ccc(N4CCN(C)CC4)cc3)nc2N(c2cccc3ccccc23)C1=O ; Cc1cccc(C)c1N1Cc2cnc(Nc3ccc(N4CCN(C)CC4)cc3)nc2N(c2cccc3cc4ccccc4cc23)C1=O]

and pharmaceutically acceptable salts of the above compounds.

A particularly preferred compound is:

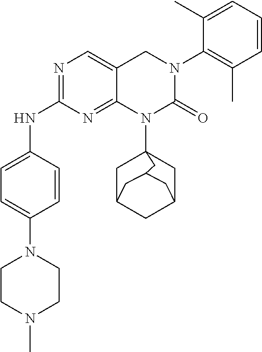 [SMILES: Cc1cccc(C)c1N1Cc2cnc(Nc3ccc(N4CCN(C)CC4)cc3)nc2N(C23CC4CC(CC(C4)C2)C3)C1=O]

and pharmaceutically acceptable salts thereof.

### Definitions

The abbreviations used herein have their conventional meaning within the chemical and biological arts. The chemical structures and formulae set forth herein are constructed according to the standard rules of chemical valency known in the chemical arts.

The term “carbon alicyclic” means, unless otherwise stated, cyclic versions of “alkyl”, which are not aromatic but may contain one or more endocyclic carbon-carbon double bonds. In preferred aspects however, a carbon alicyclic will not contain any endocyclic carbon-carbon multiple bonds. A carbon alicylic moiety includes a monocyclic or multi-ring group such as a bicyclic, tricyclic or other ulticyclic cycloalkyl ring system. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, adamantyl, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In addition, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In certain embodiments, the bridged or fused bicyclic carbon alicylic moiety is attached to the parent molecular moiety through any carbon atom contained within the monocyclic alicyclic ring. In certain embodiments, the fused bicyclic carbon alicylic moiety is a 5 or 6 membered monocyclic cycloalkyl ring fused to either a phenyl ring, a 5 or 6 membered monocyclic cycloalkyl.

The term “heteroalicyclic” or “heterocyclic” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, P, and S where the ring is saturated or unsaturated, but not aromatic. The 3 or 4 membered ring contains 1 heteroatom selected from the group consisting of O, N, P, and S. The 5 membered ring can contain zero or one double bond and one, two or three heteroatoms selected from the group consisting of O, N, P, and S. The 6 or 7 membered ring contains zero, one or two double bonds and one, two or three heteroatoms selected from the group consisting of O, N, P, and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups (which malso may be refered to as alkenyl) include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. Consistent with the below discussion of “alkenyl,” an alkenyl may include more than one double bond and/or one or more triple bonds in addition to the one or more double bonds. Consistent with the below discussion of “alkynyl,” an alkynyl may include more than one triple bond and/or one or more double bonds in addition to the one or more triple bonds. In certain aspects, an alkyl group suitably has 1 to 10 carbon atoms, 1 to 8 carbon atoms, or 1, 2, 3, 4, 5 or 6 carbon atoms.

The term “carbocyclic aryl” as used herein refers to an aromatic group where each aromatic ring atom is carbon and includes for example phenyl, naphthyl, anthracenyl, acenaphthyl, biphenyl, indene, indane, 1,2-dihydronapthalene, 1,2,3,4-tetrahydronapthalene, among others.

The term “heteroaromatic group” as used herein refers to an aromatic group where at least one aromatic ring atom is other than carbon (and may be for example N, O or S). Heteroaromatic groups include include for example pyridyl, furanyl, pyrrole, thiophene, furan, imidazole, pyrazole, oxazole, isoxazole, thiazole, isothiazole, triazole, furanzan, oxadiazole, thiadiazole, dithiazole, terazole, pyran, thiopyran, diazine, oxazine, thiazine, dioxine, dithine, and triazine, among others.

“Alkoxy” refers to a radical of the formula —ORₐ where Rₐ is an alkyl as discussed above and suitably having 1-10 carbon atoms, or 1, 2, 3, 4, 5, 6, 7 or 8 carbon atoms. Examples of alkoxy groups include without limitation —O-methyl (methoxy), —O-ethyl (ethoxy), —O-propyl (propoxy), —O-isopropyl (iso propoxy) and the like.

“Alkylthio” refers to a radical of the formula —SRₐ where Rₐ is an alkyl radical as defined above containing one to twelve carbon atoms, at least 1-10 carbon atoms, at least 1-8 carbon atoms, at least 1-6 carbon atoms, or at least 1-4 carbon atoms

“Sulfone” refers to a —S(O)₂— group in which a hexavalent sulfur is attached to each of the two oxygen atoms through double bonds and is further attached to two carbon atoms through single covalent bonds.

“Alkylsulfone” refers to a sulfone group linked to an alkyl group, for example a radical of the formula —S(O)₂(C₁₋₆alkyl).

In certain aspects, “alkenyl” refers to an unsaturated alkyl group having at least one double bond and from two to twelve carbon atoms (C₂-C₁₂ alkenyl), from two to eight carbon atoms (C₂-C₈ alkenyl) or from two to six carbon atoms (C₂-C₆ alkenyl), and which is attached to the rest of the molecule by a single bond, e.g., ethenyl, propenyl, butenyl, pentenyl, hexenyl, and the like

In certain aspects, “alkynyl” refers to an unsaturated alkyl group having at least one triple bond and from two to twelve carbon atoms (C₂-C₁₂ alkynyl), from two to ten carbon atoms (C₂-C₁₀ alkynyl) from two to eight carbon atoms (C₂-C₈ alkynyl) or from two to six carbon atoms (C₂-C₆ alkynyl), and which is attached to the rest of the molecule by a single bond, e.g., ethynyl, propynyl, butynyl, pentynyl, hexynyl, and the like.

“Amino” as used herein, and unless otherwise specified, refers to —NH₂.

“Alkylamino” as used herein, and unless otherwise specified, refers to an —NR′(R″) radical R′ and R″ may be hydrogen or substituted or unsubstituted alkyl as defined herein. In some embodiments, alkylamino is C₁-C₆-alkyl-amino such as is methylamino, ethylamino, n-, iso-propylamino, or n-, iso-, tert-butylamino. or methylamino-N-oxide, and the like.

The symbol “ [symbol] ” denotes the point of attachment of a chemical moiety to the remainder of a molecule or chemical formula.

A “substituted” moiety including a substituted R¹, R², R³, R⁴, R⁵ or R⁶ group of any of the above Formulae (I), (II), (III), (V) or (V) refers to a group that is substituted at one or more available by a non-hydrogen substituent such as hydroxyl, halogen, —CCl₃, —CBr₃, —CF₃, —CI₃, CHCl₂, —CHBr₂, —CHF₂, —, CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCL₃, —OCF₃, —OC Br₃, —OCI₃, —OCHCL₂, —OCHBr₂, —OCHF₂, —OCH₂Cl, —OCH₂Br, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), which may be further optionally substituted.

The “salt-inducible kinase” or “SIK” as used herein includes a kinase family of seine/threonine kinase that may play a role in signal transduction, for example, by controllin phosphorylation and subcellular localization of transcriptional regulatory factors (e.g., histone deacetylases (HDACs) and cAMP-regulated transcriptional coactivators (CRTCs)). The SIKs may control gene expression in response to extracellular cues (e.g., dietary salt intake in the adrenal gland) that increase intracellular levels of cAMP. Exemplary SIK may include, but not be limited to, SIK1 (Unipro ID, P57059, Q60670, Q9R1U5); SIK2 (UniPro ID: Q9H0K1, Q8CFH6, or Q9IA88); SIK1B (UniPro ID: A0A0B4J2F2) or the like.

The term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation). A “SIK inhibitor” is a compound that negatively affects (e.g. decreases) the activity or function of SIK relative to the activity or function of SIK in the absence of the inhibitor.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The terms “a” or “an,” as used in herein means one or more. In addition, the phrase “substituted with a[n],” as used herein, means the specified group may be substituted with one or more of any or all of the named substituents.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the present compounds, including any of those of Formulae (I), (II), (III), (IV), (V) or (VI) of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The present compounds and compositions preferably may be delivered transdermally, by a topical route, or formulated as applicator sticks, solutions, suspensions, emulsions, gels, creams, ointments, pastes, jellies, paints, powders, and aerosols. In one embodiment, the compound having a structure of any one of Formulae (I), (II), (III), (IV), (V) or (VI) is co-administered with a sunscreen.

The terms “disease” or “condition” refer to a state of being or health status of a patient or subject capable of being treated with the compounds or methods provided herein. In one embodiment, the “disease” or “condition” is an inflammatory disease or condition.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Patient” or “subject in need thereof” refers to a living organism suffering from or prone to a disease or condition that can be treated by administration of a pharmaceutical composition as provided herein. Non-limiting examples include humans, other mammals. In preferred embodiments, a patient or subject is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined by in vitro analyses, such as cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be safe or effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “−fold” increase or decrease. For example, a therapeutically effective amount can have at least a 1.2-fold, 1.5-fold, 2-fold, 5-fold, or more effect over a control.

In one embodiment, an “increase” refers to an amount of melanin production that is at least about 0.05 fold more (for example 0.1, 0.2, 0.3, 0.4, 0.5, 1, 5, 10, 25, 50, 100, 1000, 10,000-fold or more) than the amount of melanin production compared to a reference level (e.g., a subject having normal melanin production or a subject suffering from a disorder of melanin production). “Increased” as it refers to an amount of melanin production also means at least about 5% more (for example 5, 6, 7, 8, 9, 10, 15, 20, 25, 30, 35, 40, 45, 50, 55, 60, 65, 70, 75, 80, 85, 90, 95, 99 or 100% more) than the amount of melanin production compared to a reference level (e.g., a subject having normal melanin production or a subject suffering from a disorder of melanin production). Amounts can be measured according to methods known in the art for determining amounts of melanin.

In one embodiment, an “increase” also refers to an amount of DNA stability that is at least about 0.05 fold more (for example 0.1, 0.2, 0.3, 0.4, 0.5, 1, 5, 10, 25, 50, 100, 1000, 10,000-fold or more) than the level of DNA stability compared to a reference level (e.g., an untreated subject). “Increased” as it refers to an amount of DNA stability also means at least about 5% more (for example 5, 6, 7, 8, 9, 10, 15, 20, 25, 30, 35, 40, 45, 50, 55, 60, 65, 70, 75, 80, 85, 90, 95, 99 or 100% more) than the amount of DNA stability compared to a reference level (e.g., an untreated subject). Amounts can be measured according to methods known in the art for determining DNA damage (e.g., TUNEL assays and thymidine dimer detection).

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As discussed, pharmaceutical compositions are also provided that comprise one or more of the present compounds, including one or more compounds of Formulae (I), (II), (III), (IV), (V) or (VI) and a pharmaceutically acceptable excipient. The pharmaceutical compositions may include one or more compounds of Formulae (I), (II), (III), (IV) (V) or (VI) in a therapeutically effective amount (e.g., a therapeutically effective amount).

In addition, the pharmaceutical composition of the present invention may be manufactured with additional pharmaceutically acceptable carrier for each formulation. The type of the carrier that can be used in the present invention is not particularly limited, any carrier conventionally used in the area of industry and pharmaceutically acceptable may be used.

Saline, sterilized water, IV fluids, buffer saline, albumin injection solution, dextrose solution, maltodextrin solution, glycerol, ethanol are non-limiting examples of the usable carriers. These carriers may be used alone or in combination of two or more. The carrier may include a non-naturally occurring carrier. If necessary, other conventionally used additives like an antioxidant, and/or a buffer

A pharmaceutical composition suitably may be in the form of a spray or liquid wash or other formulation for topical application such as a lotion, cream, ointment, paste, gel, foam, or any other physical form as a carrier generally known for topical administration. Such thickened topical formulations are particularly advantageous because the formulations adhere to the area of the skin on which the material is placed, thus allowing a localized high concentration of one or more of the present compounds to be introduced to the particular area. For example, paraffin- and lanolin-based creams are generally known in the art. Other thickeners, such as polymer thickeners, may be used. The formulations may also comprise one or more of the following: water, preservatives, active surfactants, emulsifiers, anti-oxidants, or solvents.

Pharmaceutical composition may be formulated for a variety of other administration routes such as nasal, oral, parenteral, intramuscular, intra-articular, intravenous, subcutaneous, or transdermal administration. Suitable pharmaceutical compositions may be formulated with for example a diluent, a dispersant, a surfactant, a bonding agent, a lubricant to make an injection solution like aqueous solution, or as a suspension, emulsion, and as pills, capsules, granules or tablets, and the like.

As discussed, kits are also provided. For instance, one or more compounds of any one of Formulae (I), (II), (III), (IV), (V) or (VI) suitably can be packaged in suitable containers labeled, for example, for use as a therapy to treat a subject suffering from pigmentation disorders, unevenness in skin tone, hypopigmentation, inflammatory dermatoses including rosacea, vitiligo or other skin-related disorders or diseases. In addition, an article of manufacture or kit further may include, for example, packaging materials, instructions for use, delivery devices, for treating or monitoring the specified condition.

The kit may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

As discussed, methods are provided for treating a disease or disorder, such as an inflammatory disease or disorder, associated with salt-inducible kinases (SIK) by administering a compound, or a pharmaceutical composition including a compound to a subject. Preferably, the compound may have a structure of any one of Formulae (I), (II), (III), (IV), (V) or (VI). The method may suitably include administering an effective amount (e.g., therapeutically effective amount) of the compound. In preferred aspects, the methods are to treat or prevent pigmentation disorders, unevenness in skin tone, hypopigmentation, inflammatory dermatoses including rosacea, vitiligo or other skin-related diseases or disorders suitably for a subject that is suffering from or susceptible to such diseases or disorders. As discussed, the subject suitably may be a male or female human.

The subject may be suffering from or susceptible to vitiligo, which is a disorder characterized by the appearance on the skin of white patches associated with a pigmentation defect.

The subject may be suffering from or susceptible to any of erythematotelangiectatic rosacea (subtype 1 rosacea), papulopustular rosacea (subtype 2 rosacea), phymatous rosacea (subtype 3 rosacea) and/or ocular rosacea (subtype 4 rosacea). In some embodiments, the treatment methods may further comprise a step of identifying and selecting the subject suffering from rosacea, including a particular sub-type of rosacea, or other skin-related disease or disorder. In subjects suffering from erythematotelangiectatic rosacea (subtype 1 rosacea), a subject may exhibit redness and flushing of skin and visible blood vessels. In subjects suffering from papulopustular rosacea (subtype 2 rosacea), a subject may exhibit redness, swelling and acne-like breakouts. In subjects suffering from phymatous rosacea (subtype 3), a subject may exhibit thickening of facial or other skin and the skin may develop a bumpy texture. In subjects suffering from ocular rosacea (subtype 4), a subject may exhibit bloodshot and watery eyes, eyes that feel gritty, dry, itchy eyes, diminished vision. The identified and selected subject then may be treated with a therapeutic compound or composition as disclosed herein.

In some embodiments, the treatment methods may provide methods for increasing skin pigmentation and/or reducing the risk of skin cancer in a subject in need thereof. The present disclosure provides methods for increasing skin pigmentation for cosmetic purposes. In certain embodiments, provided herein are methods of increasing the appearance of skin darkening in a subject in need thereof using the described compounds (e.g., via topical administration of the described compounds).

In certain embodiments, the present disclosure provides methods of increasing the appearance of skin pigmentation in a subject, the methods comprising administering topically to the subject's skin a compound having a structure of any one of Formulae (I), (II), (III) (IV), (V) or (VI) or a pharmaceutically acceptable salt, solvate, hydrate, tautomer, or stereoisomer thereof, or a pharmaceutical composition thereof.

In certain embodiments, the present disclosure provides methods of treating polymorphic light eruption (e.g., sun hypersensitivity). The present disclosure provides methods of inducing eumelanin synthesis. The present disclosure provides methods of inducing melanosomal maturation, export, and localization.

In certain embodiments, provided are methods of reversibly increasing skin pigmentation and/or reducing the risk of skin cancer in a subject in need thereof, the method comprising administering topically to the subject's skin an effective amount of a compound having a structure of any one of Formulae (I), (II), (III), (IV), (V) or (VI), or a pharmaceutical composition thereof. In certain embodiments, provided is a method of increasing skin pigmentation and/or reducing the risk of skin cancer by topically administering the compounds described herein to a subject's skin on a body part. In certain embodiments, the body part is the face of the subject. In certain embodiments, the body part is the neck of the subject. In certain embodiments, the body part is the chest of the subject. In certain embodiments, the body part is the back of the subject. In certain embodiments, the skin on the body part is skin on the arms of the subject. In certain embodiments, the skin on the body part is skin on the legs of the subject. In certain embodiments, the skin is on the torso of the subject.

In certain embodiments, the present invention relates to the cosmetic and/or dermatological use of a compound having a structure of any one of Formulae (I), (II), (III), (IV), (V) or (VI), or a pharmaceutical composition thereof, for coloring and/or pigmenting the skin and/or body hair and/or head hair. Applications are suitable for improving pigmentation imperfections and disorders, for instance the appearance of white hairs in human beings (canities or natural whitening of hair) which can be either a visible manifestation of the aging process (senile canities), or linked to a genetic predisposition. The pigmentation of head hair and of body hair requires the presence of melanocytes in the bulb of the hair follicle. It is now accepted that canities is associated with a decrease in the amount of melanin in the hair shaft. Since maintaining a constant coloration of the head of hair is a sizeable aspiration, it is therefore desirable to be able to combat the appearance of these visible signs of aging, i.e. to maintain or re-establish the coloration of body hair and/or of head hair.

Preferred compounds can be suitably prepared in accordance with the following Scheme 1:

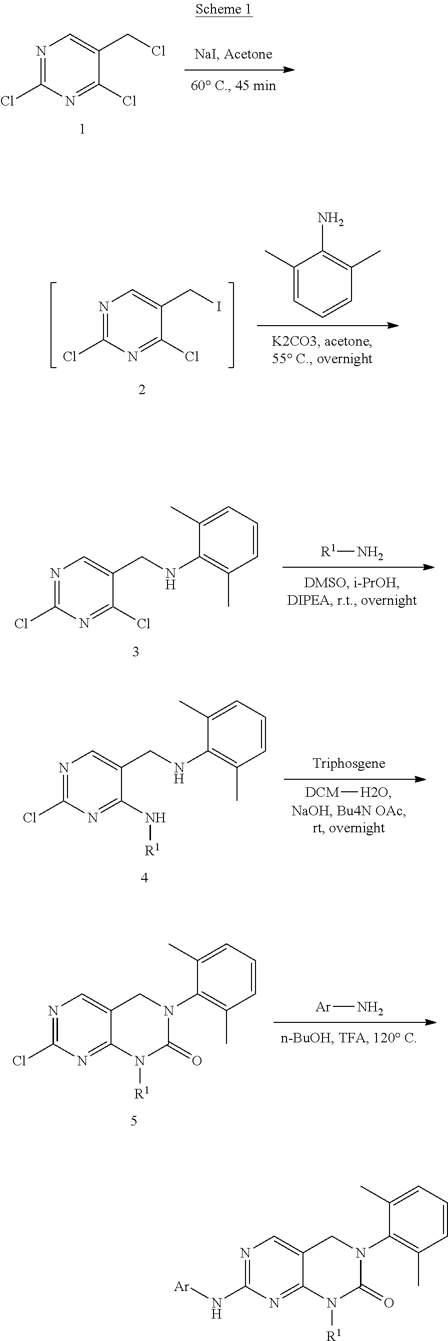

Varying Ring A (Formula (I)) groups of compounds of Formula (I) through (VI) above can be provided by use of an appropriate amine (other than the above depicted 2,6-dimethylphenylamine) in the step to yield compound 3 above such as 2-methyl-6-methoxyphenylamine, 2-methyl-6-ethoxyphenylamine, 2-methyl-6-hydroxyphenylamine, 2-methyl-6-chlorophenylamine, 2,6-di(trifluoromethyl)phenylamine, 2,4-dimethylphenylamine, 2-(—SO₂CH₃)-6-methylphenylamine, and the like.

Varying R¹—NH₂ compounds can be utilized to incorporate a desired R¹ in the compound at step 4, such as adamantyl-amine.

The following non-limiting examples are illustrative.

### Examples 1-4: Compound Syntheses

### Example 1: Synthesis of Compound 7

### Part 1: Synthesis of N-((2,4-dichloropyrimidin-5-yl)methyl)-2,6-dimethylaniline (Compound 3, Scheme 2)

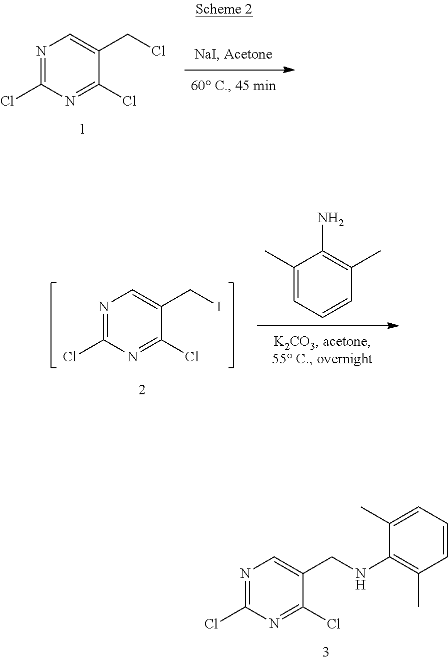

2,4-dichloro-5-(chloromethyl)pyrimidine (1) (40 g) were added in one portion to a solution of sodium Iodide (30.6 g) in acetone (244 mL) and the mixture was stirred until clear solution was obtained (intermediate (2) was not isolated). The mixture was stirred at room temperature for 15 min and then for 45 min at 60° C. under reflux under nitrogen atmosphere. The mixture was cooled and diluted in acetone (366 mL). Then, 2-6 dimethylaniline (24.9 g) and potassium carbonate (83.9 g) were added and the mixture was heated to 55° C. under reflux and nitrogen atmosphere.

The reaction mixture was poured on to ice-water and extracted with ethyl acetate. The organic layer was washed with a sodium thiosulfate aqueous solution, brine, separated, dried over anhydrous MgSO₄, filtered and the solvent was removed under reduced pressure to give a crude solid. The solid was triturated with ethyl acetate, cooled to 0° C. and filtered to afford N-((2,4-dichloropyrimidin-5-yl)methyl)-2,6-dimethylaniline (3) (46.8 g) as a white solid.

### Part 2: Synthesis of N-((3s,5s,7s)-adamantan-1-yl)-2-chloro-5-(((2,6-dimethylphenyl)amino)methyl)pyrimidin-4-amine (Compound 4, Scheme 3)

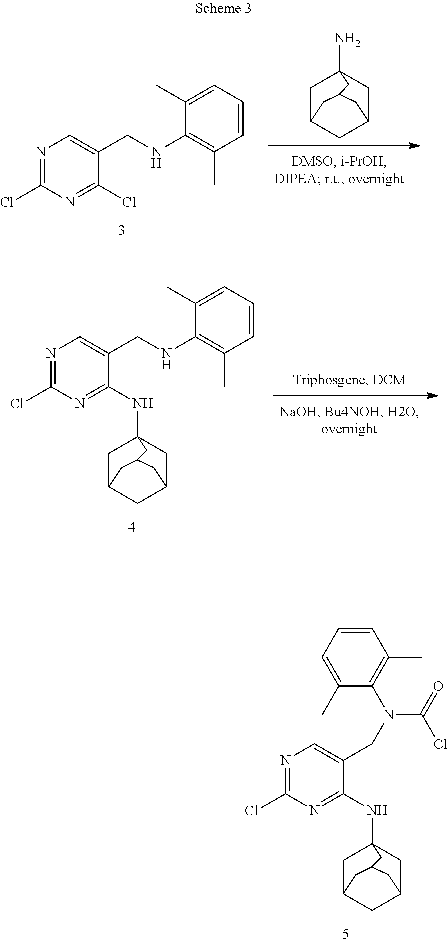 [SMILES: Cc1cccc(C)c1NCc1cnc(Cl)nc1Cl ; NC12CC3CC(CC(C3)C1)C2 ; Cc1cccc(C)c1NCc1cnc(Cl)nc1NC12CC3CC(CC(C3)C1)C2 ; Cc1cccc(C)c1N(Cc1cnc(Cl)nc1NC12CC3CC(CC(C3)C1)C2)C(=O)Cl]

N-((2,4-dichloropyrimidin-5-yl)methyl)-2,6-dimethylaniline (3)(10 g) was dissolved in THF (106 mL). Then, di-isopropylethylamine (24.6 mL) and 1-adamantamine (10.7 g) were added to the mixture and stirred at reflux for 48 h. The mixture was stirred at 80° C. in a sealed tube overnight. Ethyl acetate was added and washed with water (×3). The separated organic layer was dried over anhydrous. MgSO₄, filtered and solvent removed under reduced pressure. The crude solid was purified by flash column chromatography on silica gel (heptane/ethyl acetate (85:15). the desired fractions were collected and evaporated under reduced pressure affording N-((3s,5s,7s)-adamantan-1-yl)-2-chloro-5-(((2,6-dimethylphenyl)amino)methyl)pyrimidin-4-amine (4) (7.49 g) as a white solid.

### Part 3. Synthesis of ((4-(((3s,5s,7s)-adamantan-1-yl)amino)-2-chloropyrimidin-5-yl)methyl)(2,6-dimethylphenyl)carbamic chloride (Compound 5, Scheme 3)

Triphosgene (7.9 g) was added to a solution of N-((3s,5s,7s)-adamantan-1-yl)-2-chloro-5-(((2,6-dimethylphenyl)amino)methyl)pyrimidin-4-amine (4)(7.09 g) dissolved in CH2Cl2 (212 mL) and the mixture was stirred 1 h. A solution of sodium hydroxide (14.3 g) and tetrabutyl ammonium hydroxide (40% in water) (1.5 g) in water (212 mL) was added dropwise at 0° C. to the reaction and stirred overnight. The organic and aqueous layers were separated and the organic phase was dried over anhydrous. MgSO₄, filtered and removed under reduced pressure. The product was diluted with CH₂Cl₂, washed with saturated NaHCO₃ (×2) and water. The organic layer was separated and dried over anhydrous MgSO₄, filtered and the solvent removed under reduced pressure. The crude was purified by flash column chromatography on silica gel eluting with CH₂Cl₂. The desire fractions were collected and evaporated under reduce pressure affording ((4-(((3s,5s,7s)-adamantan-1-yl)amino)-2-chloropyrimidin-5-yl)methyl)(2,6-dimethylphenyl)carbamic chloride (5) (7.9 g) as a white solid.

### Part 4. Synthesis of 1-((3s,5s,7s)-adamantan-1-yl)-7-chloro-3-(2,6-dimethylphenyl)-3,4-dihydropyrimido[4,5-d]pyrimidin-2(1H)-one (Compound 6, Scheme 4)

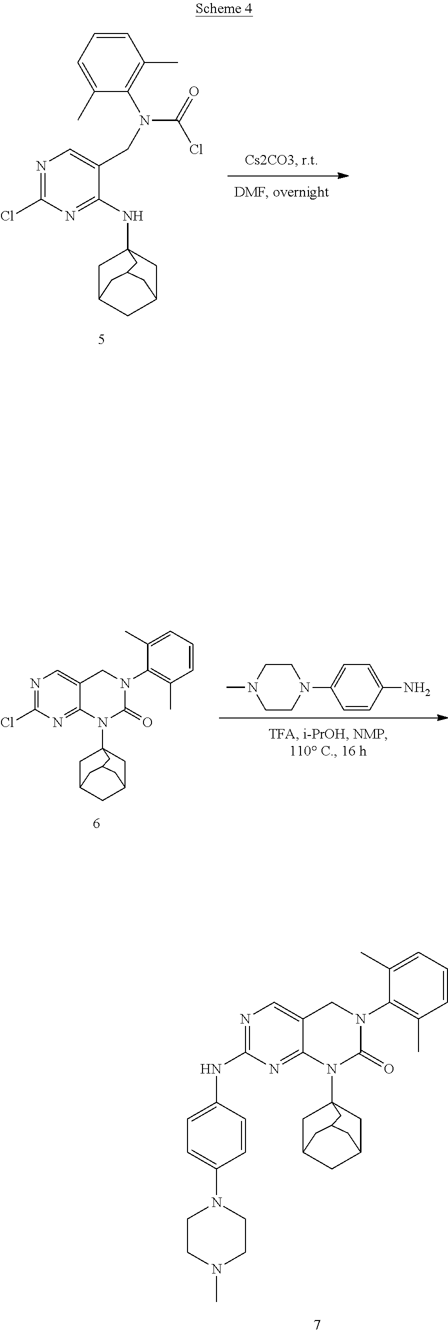

Cs₂CO₃ (8.4 g) was added to a solution of 44-4(3s,5s,7s)-adamantan-1-yl)amino)-2-chloropyrimidin-5-yl)methyl)(2,6-dimethylphenyl)carbamic chloride (7.9 g) (Compound 5) in dry DMF under nitrogen. The mixture was stirred at room temperature overnight. The mixture was then diluted with CH2Cl2 and washed with water (×2) and brine. The organic layer was separated and dried over anhydrous MgSO₄, filtered and the solvent was removed under reduced pressure to yield 1-((3s,5s,7s)-adamantan-1-yl)-7-chloro-3-(2,6-dimethylphenyl)-3,4-dihydropyrimido[4,5-d]pyrimidin-2(1H)-one (7.0 g) (Compound 6) which was used in the next step without purification.

### Part 5. Synthesis of 1-((3s,5s,7s)-adamantan-1-yl)-3-(2,6-dimethylphenyl)-7-((4-(4-methylpiperazin-1-yl)phenyl)amino)-3,4-dihydropyrimido[4,5-d]pyrimidin-2(1H)-one (Compound 7, Scheme 4)

4-(4-methylpiperazin-1-yl)aniline (5.5 g) and trifluoro acetic acid (6.3 mL) were added to a solution of 1-((3s,5s,7s)-adamantan-1-yl)-7-chloro-3-(2,6-dimethylphenyl)-3,4-dihydropyrimido[4,5-d]pyrimidin-2(1H)-one (6) (7.2 g) and N methyl pyrrolidone (6.2 mL) in isopropanol (94 mL). The mixture was stirred in a sealed tube at 110° C. overnight. Ethyl acetate was added and the mixture washed with an aqueous sodium bicarbonate solution and brine. The separated organic layer was dried over anhydrous MgSO4, filtered and the solvent was removed under reduced pressure. The crude mixture was purified by flash column chromatography on silica gel eluting with a gradient of CH2Cl2: MeOH (100:0 to 70:30). The desired fractions were collected and the solvent removed under reduced pressure. The desired compound (7) was recrystallized from CH2Cl2-Acetonitrile and filtered to yield 1-((3s,5s,7s)-adamantan-1-yl)-3-(2,6-dimethylphenyl)-7-((4-(4-methylpiperazin-1-yl)phenyl)amino)-3,4-dihydropyrimido[4,5-d]pyrimidin-2(1H)-one (6.14 g) as a white solid. This compound (7) is sometimes referred to herein as SLT-008.

Analysis: LCMS (rt 2.647 min, m/z 578.4).

Melting temperature: 283.5° C.

¹H NMR (400 MHz, CDCl₃) δ 7.96 (s, 1H), 7.51-7.44 (m, 2H), 7.15-7.04 (m, 3H), 6.94 (d, J=9.0 Hz, 2H), 6.88 (s, 1H), 4.18 (s, 2H), 3.25-3.19 (m, 4H), 2.67 (s, 4H), 2.55 (d, J=1.8 Hz, 6H), 2.42 (s, 3H), 2.16 (s, J=6.0 Hz, 6H), 2.12 (d, J=4.2 Hz, 3H), 1.70 (dd, J=27.8, 12.0 Hz, 6H).

### Examples 2-4

By the procedures of Example 1 above with substitution of an appropriate amine compounds in the above Scheme 1 above (i.e. amines to produce compounds corresponding to compounds 3 and 4 in Scheme 1 above), the following compounds of Examples 2-4 are prepared.

### Example 2

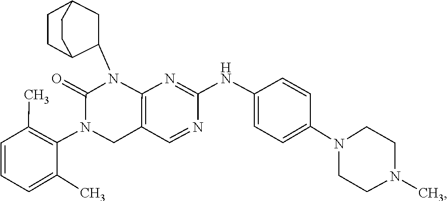 [SMILES: Cc1cccc(C)c1N1Cc2cnc(Nc3ccc(N4CCN(C)CC4)cc3)nc2N(C2CC3CCC2CC3)C1=O]

### Example 3

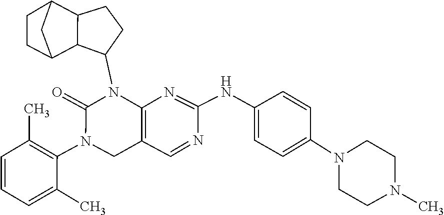 [SMILES: Cc1cccc(C)c1N1Cc2cnc(Nc3ccc(N4CCN(C)CC4)cc3)nc2N(C2CCC3C4CCC(C4)C32)C1=O]

### Example 4

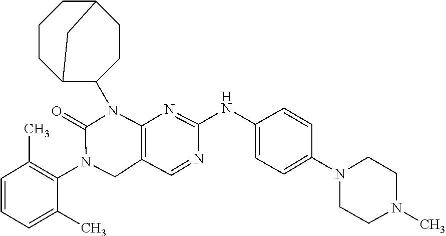 [SMILES: Cc1cccc(C)c1N1Cc2cnc(Nc3ccc(N4CCN(C)CC4)cc3)nc2N(C2CCC3CCCC2C3)C1=O]

### Example 5: Salt Induced Kinase Inhibition

The Salt Induced Kinase (SIK) inhibitor SLT-008 (Compound 7) can be optimized as topical agents capable of inducing cutaneous pigmentation.

SIK 1(h) was incubated with 8 mM MOPS pH 7.0, 0.2 mM EDTA, 100 μM AMARAASAAALARRR, 10 mM Magnesium acetate and [γ- ³³P]-ATP (45 μM). The reaction was initiated by the addition of the Mg/ATP mix. After incubation for 40 minutes at room temperature, the reaction wa stopped by the addition of phosphoric acid to a concentration of 0.5%. 10 μL of the reaction was then spotted onto a P30 filtermat and washed four times for 4 minutes in 0.425% phosphoric acid and once in methanol prior to drying and scintillation counting.

SIK2(h) was incubated with 8 mM MOPS pH 7.0, 0.2 mM EDTA, 100 μM KKKVSRSGLYRSPSMPENLNRPR, 10 mM Magnesium acetate and [γ-³³P-ATP](45 μM). The reaction was initiated by the addition of the Mg/ATP mix. After incubation for 40 minutes at room temperature, the reaction was stopped by the addition of phosphoric acid to a concentration of 0.5%. 10 μL of the reaction was then spotted onto a P30 filtermat and washed four times for 4 minutes in 0.425% phosphoric acid and once in methanol prior to drying and scintillation counting.

SIK3(h) was incubated with 8 mM MOPS pH 7.0, 0.2 mM EDTA, 100 KKKVSRSGLYRSPSMPENLNRPR, 10 mM Magnesium acetate and [γ- ³³P-ATP] (45 μM). The reaction was initiated by the addition of the Mg/ATP mix. After incubation for 40 minutes at room temperature, the reaction was stopped by the addition of phosphoric acid to a concentration of 0.5%. 10 μL of the reaction was then spotted onto a P30 filtermat and washed four times for 4 minutes in 0.425% phosphoric acid and once in methanol prior to drying and scintillation counting.

The IC50 was calculated to be 5, 8 and over 20 nanomolar for SIK1, SIK2 and SIK3 respectively.

TABLE 1
Estimated IC₅₀ Values

| Compound | Kinase | IC50 (nM) |
|---|---|---|
| SLT-008 | SIK(h) | 5 |
| SLT-008 | SIK2(h) | 8 |
| SLT-008 | SIK3(h) | >20 |

### Examples 6-10: In Vitro and Ex Vivo Skin Tissue Models Show

Pigmentation Efficacy of SLT-008 (SLT-008 designated herein as Compound 7 in Scheme 4 above, 1-((3s,5s,7s)-adamantan-1-yl)-3-(2,6-dimethylphenyl)-7-((4-(4-methylpiperazin-1-yl)phenyl)amino)-3,4-dihydropyrimido[4,5-d]pyrimidin-2(1H)-one).

### Example 6: Topical Application of SLT-008 Induces Melanin Pigmentation

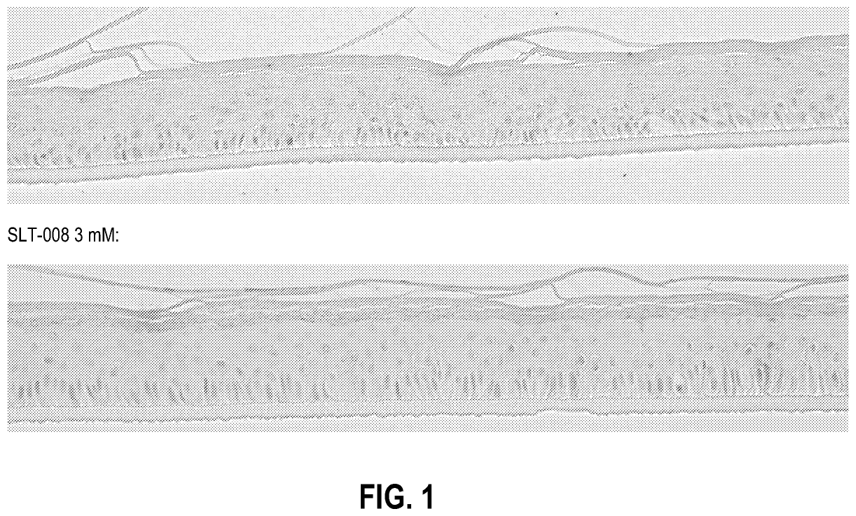

Primary human epidermal keratinocytes (hEK) were seeded onto suitable carrier matrices and cocultured with human epidermal melanocytes. SLT-8 was solubilized in PEG/Ethanol/Transcutol® at 0.3 mM, 1 mM and 3 mM and applied topically using a mesh to the surface of the models and removed after 4 hours. Test compounds were applied once daily over a period of 4 days. Untreated models and models treated with the vehicle only served as controls. UV irradiated models were used as positive controls. Fontana Masson staining was conducted to visualize pigmentation effects. Fontana-Masson staining is routinely used to visualize argentaffin substances such as melanin, argentaffin granules and some neurosecretory granules. Melanin is a nonlipid, non-hematogenous pigment. It is a brown-black pigment present normally in the hair, skin, retina, iris, and certain parts of the central nervous system. Argentaffin granules are found in carcinoid tumors. The argentaffin cell granules or melanin stain black. Nuclei stain pink-red. The cytoplasm stains pale pink. At 1 mM, and specifically at 3 mM, a single topical application of SLT-008 induced melanin pigment dots at the stratum lucidum level. At 3 mM, a single topical application also induced melanin transfer to keratinocytes and nuclear capping was observed (FIG. 1).

### Example 7: SLT-008 Pigmentation Activity on Human Living Skin Explants

The pigmentation activity of SLT-008 at two concentrations was evaluated on skin using human living skin explants with stripping.

Human skin explants of an average diameter of 11 mm (±1 mm) were prepared on an abdominoplasty from a 35-year-old Caucasian woman with a III phototype. The explants were kept alive in BEM culture medium (BIO-EC's Explants Medium) at 37° C. in a humid, 5%-CO2 atmosphere. The study was performed on human skin tissue, obtained from surgical residues in accord with the Declaration of Helsinki and the article L.1243-4 of the French Public Health Code.

On day 0, the explants of the batches “S” were stripped 5 times using scotch 3M to remove some stratum corneum layers to facilitate the penetration of the tested products. The excipient and the tested products were applied topically on the basis of 2 μl per explant (2 mg/cm²) and spread using a small spatula on day 0 (D0, after the stripping), D1, D4, D6 and D8.

The control explants T did not receive any treatment except the renewal of culture medium. The culture medium was half renewed (1 ml per well) on D0, D1, D4, D6 and D8. On D0, D1, D4, D5, D6, D7 and D8, the culture media of the irradiated explants (TUV) were replaced by HBSS (Hank's Balanced Saline Solution; 1 ml per explant). Then, the explants of the “TUV” batch were irradiated using a UV simulator Vibert Lourmat RMX 3W with a dose of 2.25 J/cm2 of UVA corresponding to 0.5 MED (minimal erythemal dose) for a phototype II donor. At the end of the UV irradiation, these explants were put back in 2 mL of BEM medium. It was determined that irradiation with UVA (TUVJ11 vs TJ11) induces no modification of cell viability. Treatment with 0.5% and 0.9% of SLT-008 compared to the stripped batch also resulted in no modification of cell viability. Irradiation with UVA (TUVJ11 vs TJ11) induced an increase of melanin synthesis in the basal layer of the epidermis.

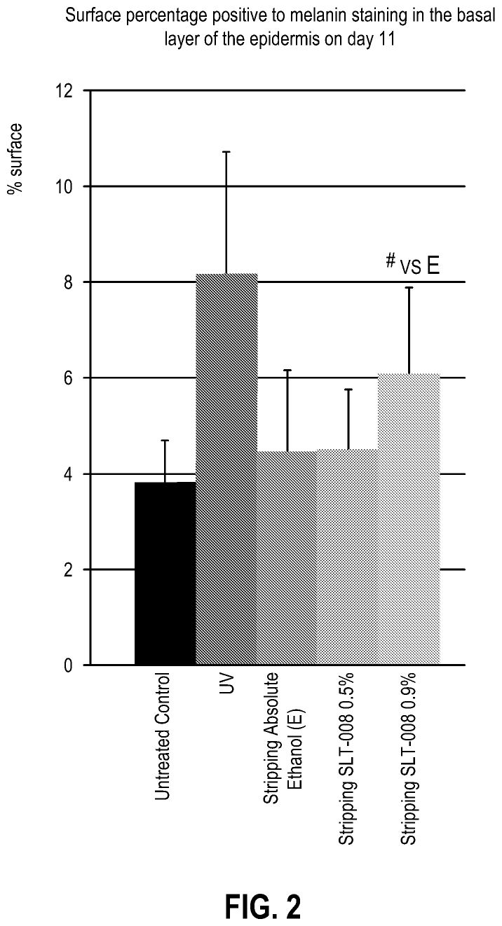

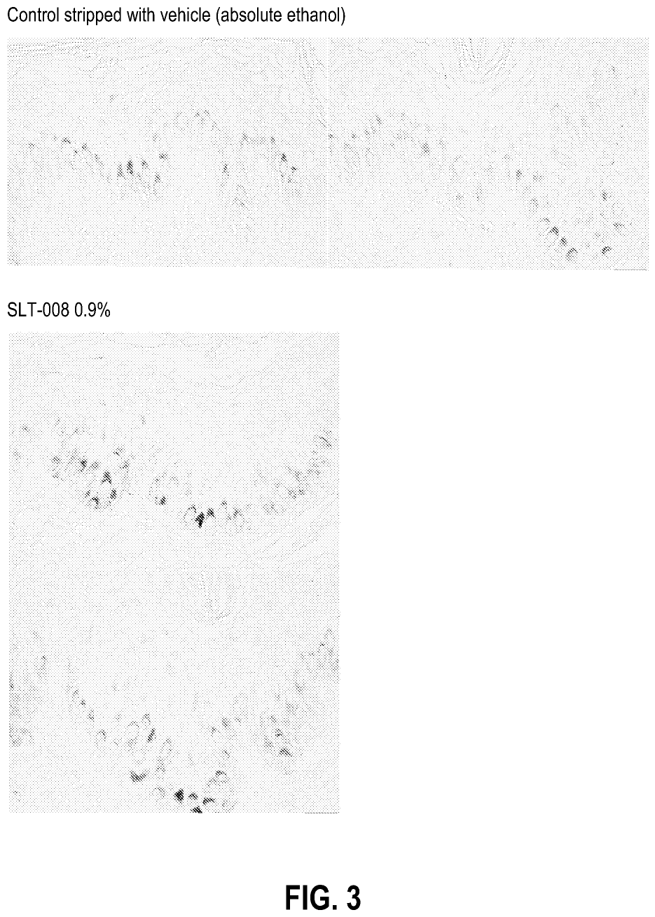

The 0.9% dose of SLT-008 induced a significant (p<0.1) melanin increase of 36% (FIG. 2). Pigmentation activity was also detected by Fontana Masson staining (FIG. 3).

### Example 8: Dose Determination and SLT-008 Systemic Exposure

Studies were undertaken to determine three non-toxic concentrations of SLT-008 that do not produce systemic cytotoxicity for Normal Human Epidermal Melanocytes (NHEMs-MP; Thermo Fisher Scientific, C1025C) in a culture medium of reconstituted epidermis.

The preliminary dose determination study was performed using cultures of NHEMs grown in M254 medium (Thermo Fisher Scientific, M254500) supplemented with Human Melanocyte Growth Supplement (HMGS; Thermo Fisher Scientific, S0025) and antibiotics (Gentamycin, Thermo Fisher Scientific, 15710049). The cells were maintained in a humidified incubator at 37° C. with a 5% CO2 atmosphere.

NHEMs were seeded in 24-well plates, 24 h before the beginning of treatment with the test compound at 5 concentrations, in triplicates (n=3). DMSO 0.003% used for the test compounds solubilization was tested in parallel. A total of 6 repeated applications in the culture medium was performed over 10 days (from day 1 until day 11 after seeding, medium was refreshed at days 4, 5, 6, 7, and 8). SDS 0.008% was used as cytotoxic positive control. At the end of treatments, cell viability was evaluated with a MTS assay (3-(4,5-dimethylthiazol-2-yl)-5-(3-carboxy-methoxyphenyl)-2-(4-sulfophenyl-2H-tetrazolium).

Next, absence of toxicity of the three doses was evaluated in reconstituted melanized epidermis by hematoxylin/eosin (H/E) staining of histologic sections.

At the end of the treatments with the test compounds, 3 tissues per dose (n=3) were fixed in 4% formaldehyde, dehydrated and paraffin embedded. Sections of 6 μm of epidermis were stained with eosin and hematoxylin (H/E). Slides were mounted with specific medium and examined with a Leica DM2000 photomicroscope coupled to a digital camera (Zeiss).

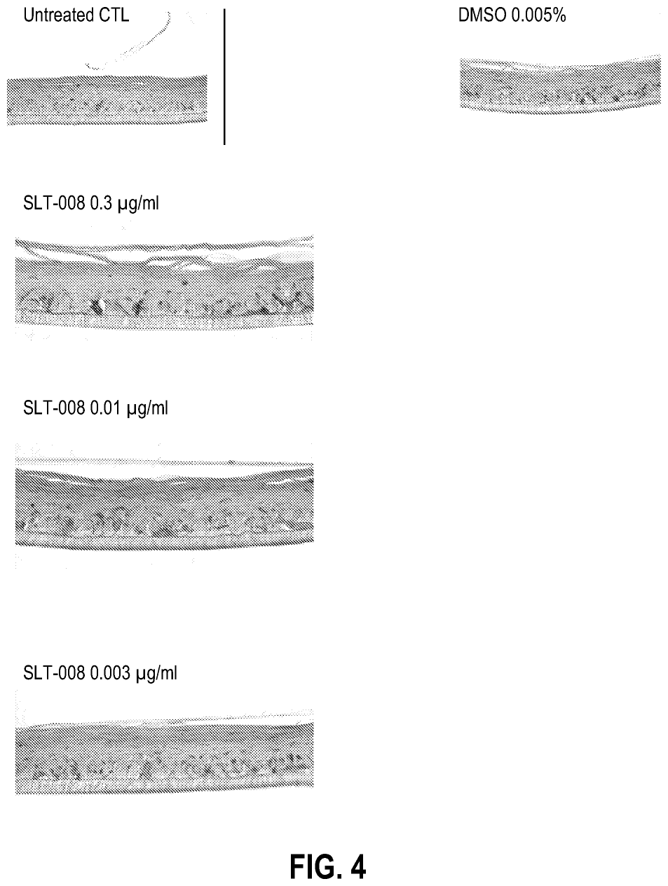

The analysis revealed no morphologic alteration after the treatments with the 3 selected doses of SLT-008 (FIG. 4).

### Example 9: LDH Release Assay

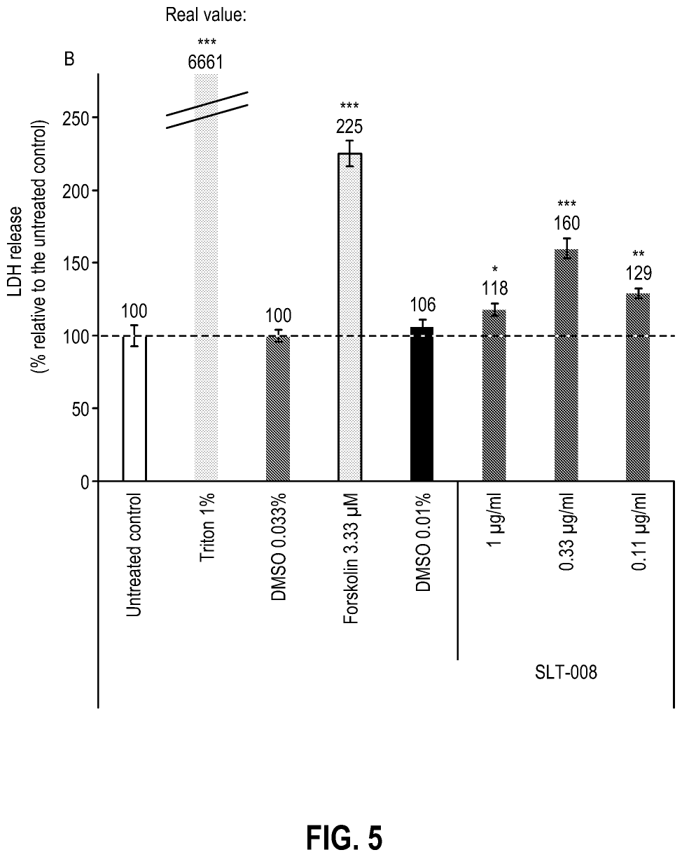

Quantification of lactate dehydrogenase (LDH) release from melanized tissue was performed at mid-treatment and at the end of treatment with the SLT-008 to ensure that the selected doses were not cytotoxic for the melanized reconstructed human epidermis. The release of LDH by the culture model was assessed after 5 and 10 days of treatments with the Cytotoxicity Detection KitPLUS (Roche-04744926001) according to the manufacturer's instructions. Briefly, culture supernatants were collected and mixed with LDH detection reactants in 96-well plates for 30 min before measuring optical density at 490 nm with a reference wavelength of 600 nm using a spectrophotometer (GloMax-Promega). This was performed in triplicates of cultures (n=3). Altogether, the application of SLT-008 for 10 days on melanized reconstructed epidermis did not induce relevant alteration of the tissue integrity. Slight and significant increase in LDH release from tissues could be observed after 10 days of treatment with the dose of 0.3 μg/ml of SLT-008, but without reaching the set cytotoxic level. The effect of these concentrations was thus studied on the tissue pigmentation (FIG. 5).

### Example 10: Melanin Content

Based on the results of dose determination studies, 0.3 μg/ml, 0.01 μg/ml and 0.0033 μg/ml doses of SLT-008 were selected for further application in a culture medium of reconstituted melanized human epidermis. The pigmentation effect studies were carried out on epidermis reconstituted with NHEKs (Normal human epidermal keratinocytes, Lonza 00192906) isolated from foreskin of 3 neonatal Caucasian donors and NHEMs-MP melanocytes.

The tissues were cultured at the air-liquid interface in Epilife medium (Fisher Scientific, MEPI500CA) containing specific supplements (with among others Human Keratinocytes Growth Factors, Fisher Scientific S0015 or S001K) and antibiotics (Gentamycin, Fisher Scientific, 15710049). They were maintained in a humid atmosphere at 37° C. with 5% CO2.

Melanocytes were co-cultured with reconstructed epidermis and cultivated at the air-liquid interface during 14 days in culture medium in a humid atmosphere at 37° C. with CO2 5%. SLT-008 was applied in the culture medium at 3 concentrations, during 10 days, from day 4 to day 14 after placement at the air/liquid interface (n=6), with 5 medium refreshes (at day 7, 8, 9, 10 and 11). In order to allow pigmentation of the epidermis, the concentration in BPE (bovine pituitary extract) in culture medium was adapted during the treatments. DMSO at 0.005%, used for the compounds solubilization was used as reference control condition. IBMX at 150 μM and forskolin at 3.33 μM were used as pro-pigmentation reference compounds to validate the experiment. An additional DMSO control at 0.05% corresponding to the solvent of the reference compounds was added as well in the experiment.

At the end of treatments (day 14), a morphology analysis was performed. The epidermis pigmentation was quantified after solvable extraction and quantified through Fontana Masson staining of histologic sections. 6 μm sections of epidermis embedded in paraffin were prepared and stained with Fontana Masson reagents. Fontana Masson staining specifically stains melanin within the epidermis. In order to visualize the epidermis in addition to the melanin, a counterstaining with hemalum/eosin was performed as well. Tissue section staining was performed in three series with one set for each test compound. Slides with sections of epidermis treated with DMSO at 0.005% were put in each set for Fontana Masson staining in order to compare the effects of the SLT-008.

Melanin deposition on 6 μm paraffin sections of each epidermis was stained by Fontana-Masson reagents (VWR-VWRK641295, VWRK641311; Klinipath-641215). Slides were mounted with specific medium and examined with a Leica DM2000 photomicroscope equipped with a digital camera (Zeiss).

For the melanin quantification, 3 pictures of each epidermis were taken and analyzed by using the Leica QWin3 software. Two measurements were taken. The first one corresponds to the global intensity of the staining, reflecting a lighter or darker melanin. The second one represents the labelled surface on the picture, and thus the area occupied by the melanin in the epidermis. These measurements were obtained by examining all cell layers of epidermis excepted the stratum corneum.

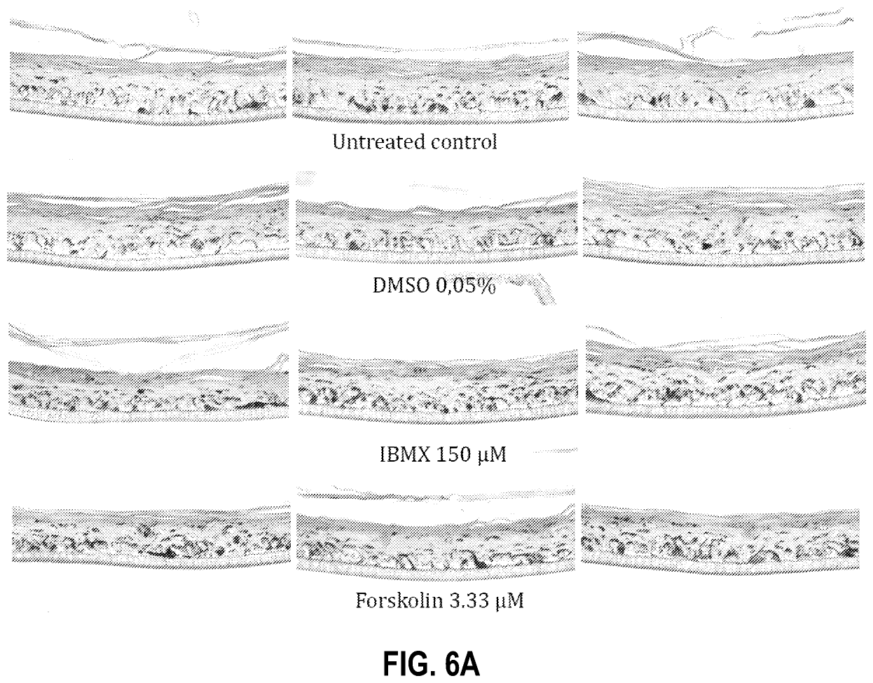

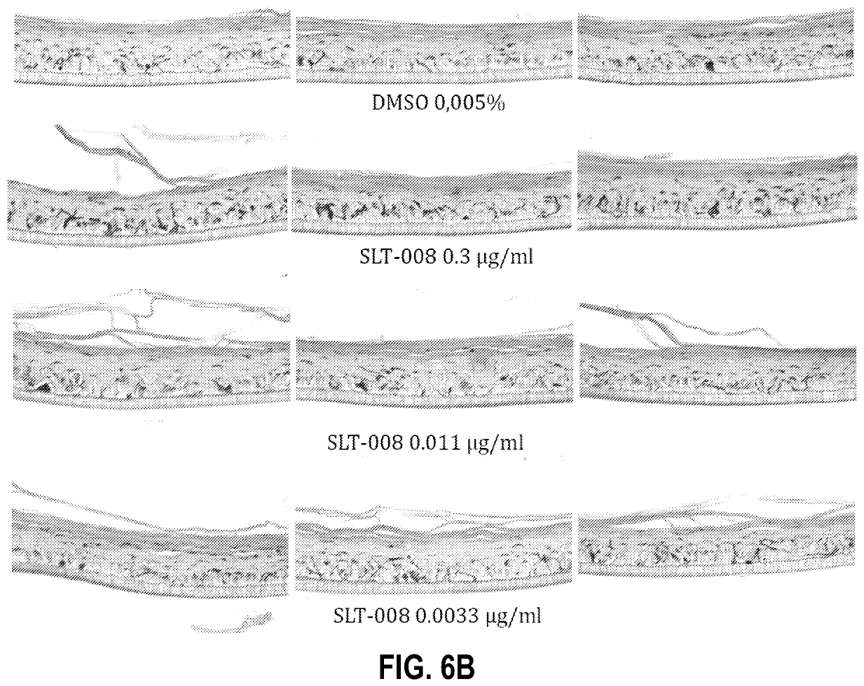

The analysis of melanin content was then observed. The analysis was performed from 9 pictures per dose (3 pictures per epidermis and 3 epidermis per condition for a total of 9 pictures). FIG. 6A shows the untreated control, the vehicle (DMSO 0.05%) and two positive controls showing a complete epidermal pigmentation as illustrated by a dendritic morphology of functional melanocytes, and the formation of melanosomes organized as supranuclear melanin caps above the keratinocyte nuclei (FIG. 6). The pigmentation was increased by forskolin (3.33 μM) or IBMX (150 μM) vs DMSO 0.05%. Forskolin and IBMX are known as epidermal inducers of melanin production and are used here as positive controls to confirm the in vitro functional pigmentation. As shown in FIG. 6B, the pigmentation was significantly increased by SLT-008 at 0.3 μg/ml vs DMSO 0.005%. The increase is observed both on melanosomes, presence of dendrites and melanin capping.

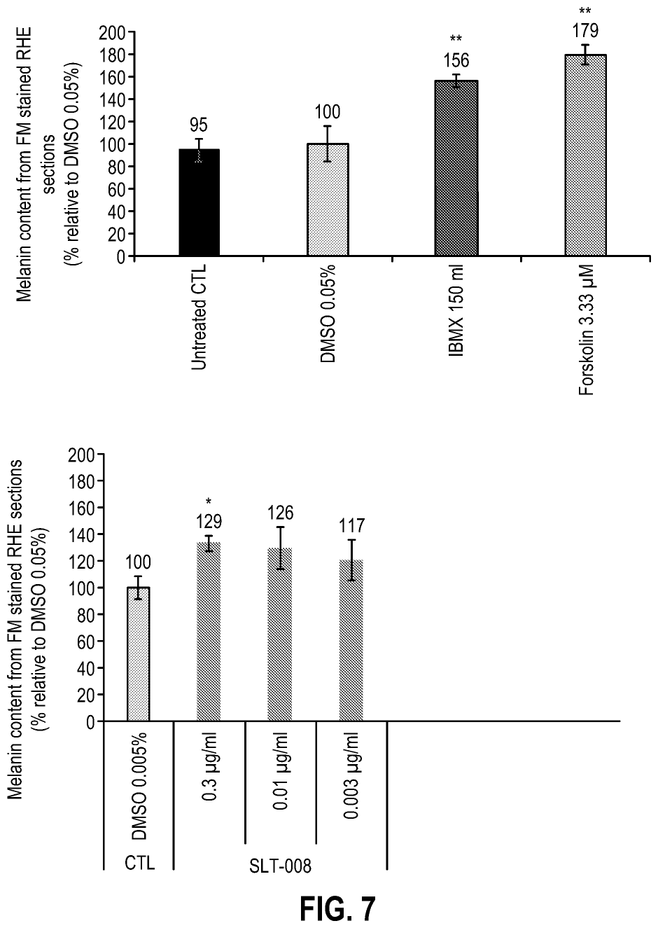

Melanin content was determined from Fontana-Masson stained sections (% relative to DMSO 0.005%). A statistical analysis (Student-t-test) was used to compare the effects of the SLT-008 vs DMSO at 0.005% and the effects of IBMX and forskolin vs DMSO at 0.05%, with 0.01<p-values<0.05 considered as significant (*), 0.001<p-values<0.01 as highly significant (**) and p-values<0.001 as very highly significant (***). Compound SLT-008 at 0.3 μg/ml induced a significant increase in melanin content as compared to their respective DMSO controls at 0.005% (FIG. 7).

Using similar methods described previously in this Example, the experiments were repeated. At the end of these treatments, tissues (n=3) were removed from their inserts and immerged in a solvable extraction solution (Perkin Elmer-NJTSRN8010060) and heated at 80° C. for 1 h. The optical density of the supernatants was measured at 490 nm with a spectrophotometer (GloMax-Promega) and the melanin content was determined by comparison with a standard curve of synthetic melanin (Sigma-M8631)

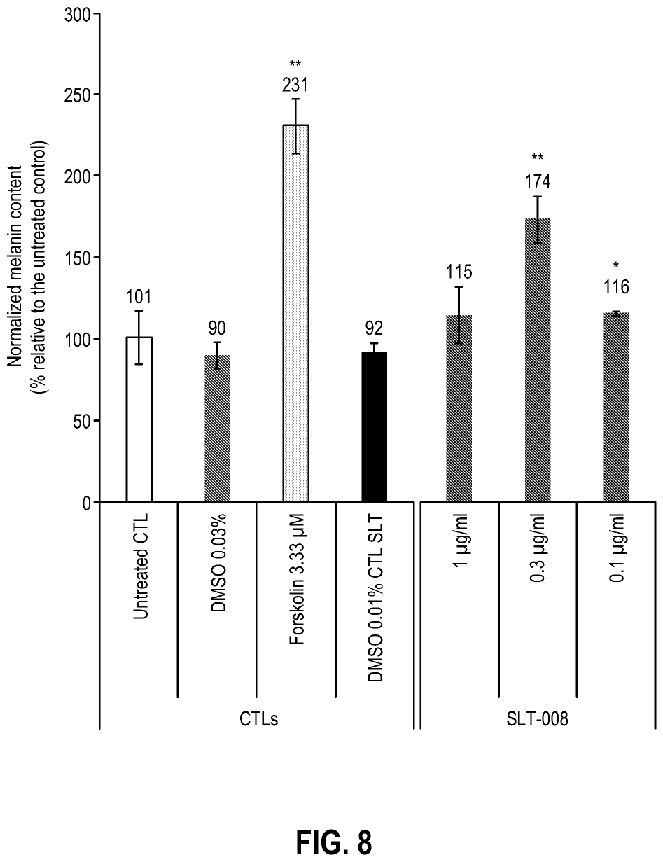

The treatments with forskolin (positive control) significantly increased the production of melanin within the epidermis, as compared to its solvent control (DMSO at 0.03%). The DMSO used as solvent at 0.03 and 0.01% did not modify the melanin content level, indicating its passive effect on pigmentation. SLT-008 increased, with a dose effect, the melanin content when applied at 1 and 0.1 μg/ml and more so, at 0.3 μg/ml (FIG. 8). Based on these studies, the systemic application of SLT-008 increased in vitro the melanin content of reconstructed epidermis tissue models observed both by Fontana Masson staining and chemical extraction quantification.

### Example 11: Stripping-induced Apoptosis DNA Protection Study

The aim of this study was to perform complementary immunostaining of TUNEL (apoptotic cell marker).

TABLE 2
Batches Analyzed in the UVA Controlled Irradiation Study:

| Batch | Designation | Explants | Sampling time |
|---|---|---|---|
| T0 | Control of the tissue | 3 | Day 0 |
| T | Non-treated control | 3 | Day 11 |
| TUV | Control UV (UVA 0.5MED) | 3 | Day 11 |
| S | Stripping | 3 | Day 11 |
| SP9 | Stripping + B (SLT-008) | 3 | Day 11 |
| | 0.5% + excipient | | |
| SP10 | Stripping + B (SLT-008) | 3 | Day 11 |
| | 0.9% + excipient | | |

TABLE 3
Experimental Schedule

| | | | | | | | | | | | |
|---|---|---|---|---|---|---|---|---|---|---|---|
| D 0 | D 1 | D 2 | D 3 | D 4 | D 5 | D 6 | D 7 | D 8 | D 9 | D 10 | D 11 |

| | | | | | | | |
|---|---|---|---|---|---|---|---|
| ▪ | ▪ | ▪ | ▪ | ▪ | ▪ | ▪ | |
| ▾▴ | ▴ | ▴ | | ▴ | | ▴ | □ |

▾ Explants sampling
▴ Product application
▪ UV-A irradiation

Histological processing: 5-μm-thick sections were made using a Leica RM 2125 Minot-type microtome, and the sections were mounted on Superfrost® histological glass slides. The microscopical observations were realized using a Leica DMLB, an Olympus BX43 or BX63 microscope. Pictures were digitized with a numeric DP72 or DP74 Olympus camera with CellSens storing software.

TUNEL assay: DNA damage was assessed on formol-fixed paraffin-embedded skin sections using a In Situ Cell Death Detection Kit (Roche, ref 11 684 817 910) with the TUNEL reagent diluted at 1:2 in PBS for 1 hour at room temperature and the POD converter diluted at 1:4, then revealed by VIP (Vector, ref. SK-4600). The staining was semi-quantified by image analysis.

Image analysis method: The images analyses was performed on all the images of the selected batch using CellSens software. Stained surfaces comparisons

The stained surface percentage (Surf %) for the treatment is compared to the untreated condition=>S vs T; SP vs S.

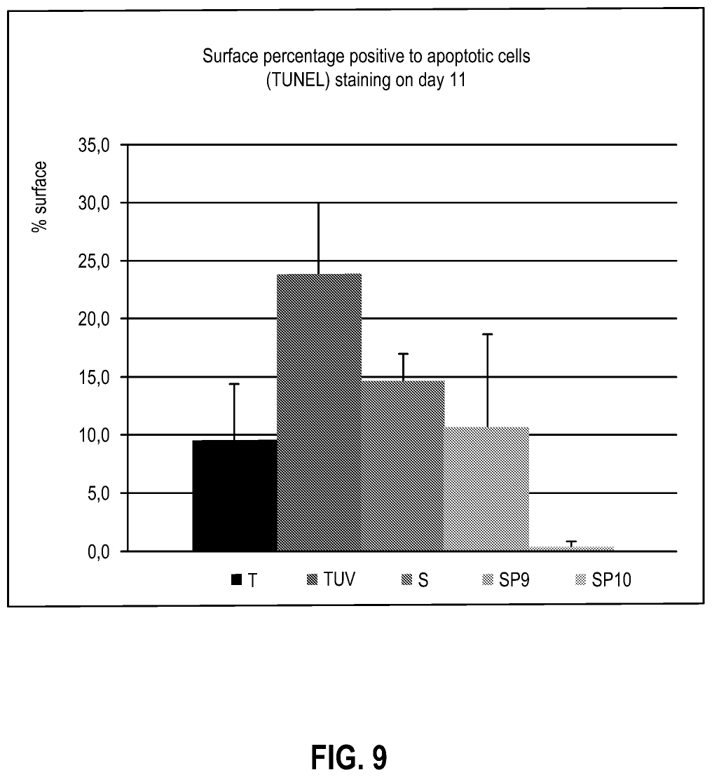

The percentage of surface occupied by TUNEL staining related to apoptotic cells in the epidermis is shown in FIG. 9. SLT-008 0.9.% in absolute ethanol significantly decreased by 97% apoptotic cell numbers after tape stripping, thereby demonstrating DNA protection.

### Example 12: UVB DNA Protection Study

The aim of this study was to evaluate the effects of a product DNA protection using living human skin explants.

TABLE 4
DNA Protection Activity (UVB)

| D 0 | D 1 | D 2 | D 3 | D 4 | D 5 |
|---|---|---|---|---|---|
| | | | | ▪ | |
| ▾▴ | | ▴ | | ▴ ▴ | ▾ |

▾ Explants sampling
▴ Product application

63 human skin explants of an average diameter of 11 mm (±1 mm) were prepared on an abdoplasty coming from a 51-year-old Caucasian woman with a II-III phototype (according to Fitzpatrick skin color classification). The explants were kept in survival in BEM culture medium (BIO-EC's Explants Medium) at 37° C. in a humid, 5%-CO₂ atmosphere.

TABLE 5
Explant Distribution

| Batch | Conditions | Explants | Sampling time |
|---|---|---|---|
| T0 | Control of the tissue | 3 | Day 0 |
| T | Non-treated control | 12 | Day 5 |
| E | Placebo | 3 | Day 10 |
| P1 | Formulation SLT-008 0.1% | 3 | Day 10 |
| P2 | Formulation SLT-008 0.5% | 3 | Day 10 |
| UVB | Control UVB | 9 | Day 5 |
| EUVB | Placebo + UVB (2 MED) | 9 | Day 5 |
| P1UVB | Formulation SLT-008 | 9 | Day 5 |
| | 0.1% + UVB (2 MED) | | |
| P2UVB | Formulation SLT-008 | 9 | Day 5 |
| | 0.5% + UVB (2 MED) | | |

DNA Protection Activity (UVB): The excipient and the tested products P1 and P2 were applied topically at a concentration of 2 μl per explant (2 mg/cm²) and spread using a small spatula on day 0 (D0), D2, D4 and D6. On D4, the excipient and the products P1 and P2 were applied twice (before and just after UV-B irradiation). The control explants (T) did not receive any treatment except the renewal of culture medium. The culture medium was half renewed (1 ml per well) on D2, D3 and D6.

DNA Protection Activity (UVB): On D4, the culture media of the irradiated explants (UVB) were replaced by HBSS (Hank's Balanced Saline Solution; 1 ml per explant). Then, the explants were irradiated using a UV simulator Vibert Lourmat RMX 3W with a dose of 0.3 J/cm² of UVB corresponding to 2 MED on a skin with a II-III phototype. At the end of the UV irradiation, the UVB treated explants were put back in 2 mL of BEM medium.

Sampling: On D0, the 3 explants from TO were collected and cut in two parts. Half was fixed in buffered formalin solution and half was frozen at −80° C. On D5, D6, D7 and D10, 3 explants from the concerned batches were collected and treated in the same way than in D0. According to the dispositions mentioned in the study plan, the days of treatments, irradiations and sampling were adjusted to the schedule of working days.

Histological processing: After fixation for 24 hours in buffered formalin, the samples were dehydrated and impregnated in paraffin using a Leica PEARL dehydration automat. The samples were embedded using a Leica EG 1160 embedding station. 5-μm-thick sections were made using a Leica RM 2125 Minot-type microtome, and the sections were mounted on Superfrost® histological glass slides. The microscopical observations were realized using a Leica DMLB, Olympus BX43 or BX63 microscope. Pictures were digitized with a numeric DP72 or DP74 Olympus camera with CellSens storing software.

Thymine dimer immunostaining: Ultraviolet light is absorbed by a double bond in thymine and cytosine bases in DNA. This added energy opens up the bond and allows it to react with a neighboring base. If the neighbor is another thymine or cytosine base, it can form a cyclobutane ring linking the two bases. These cyclobutane dimers are constrained and form a covalent crosslink in the DNA. This causes problems when the cell needs to replicate its DNA. DNA polymerase has trouble reading the dimer, since it does not fit smoothly in the active site. Thymine dimer immunostaining was performed on FFPE skin sections with a monoclonal anti-thymine dimers antibody (Kamiya, ref. MC-062, clone KTM53) diluted at 1:1600 in PBS-BSA 0.3%-Tween 20 at 0.05% and incubated 1 hour at room temperature using a Vectastain Kit Vector amplifier system avidin/biotin, and revealed by VIP, a substrate of peroxidase (Vector laboratories, Ref. SK-4600) giving a violet signal once oxidized. The staining was semi-quantified by image analysis using the software CellSens.

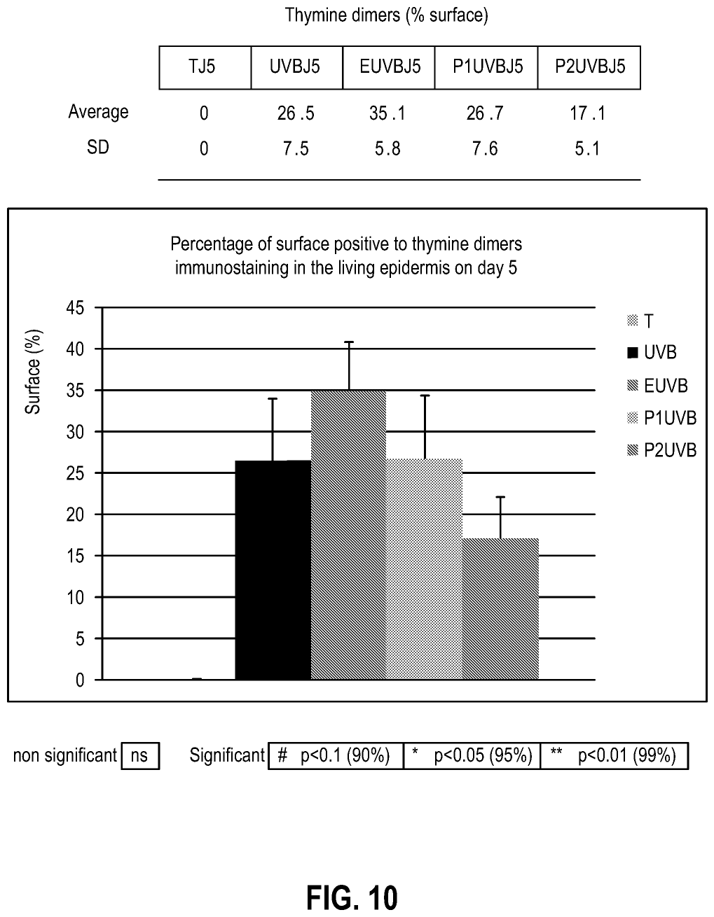

No thymine dimers were observed on unirradiated batches. The percentage of surface occupied by thymine dimers in the epidermis for all batches is shown in FIG. 10. On day 5, UVBJ5, thymine dimers represented 26.5% of the surface of the epidermis. Effect of product application on thymine dimers formation was compared to the UVBJ5. The excipient induced a significant increase of 32%, P1 induced a non-significant increase of 1%, and P2 induced a significant decrease of 35%. The effect of product application on thymine dimers formation was also compared EUVBJ5. P1 induced a significant decrease of 24% and P2 induced a significant decrease of 51%.

TUNEL Assay: DNA damaged (apoptotic cells) were stained with a in situ detection kit for cellular death (Merck, ref no. 11 684 817 910) using the TUNEL reagent diluted at 1:2 in PBS for 1 hour at room temperature and the POD converter diluted at 1:4 in PBS and then revealed by VIP (Vector Laboratories, ref: SK-4600). The staining was semi-quantified by image analysis using the software CellSens. For each sample of explants, the percentage of the region of interest covered by the staining (stained surface percentage) was determined by image analysis. The stained surface percentage (Surf %) for the treatment was compared to the untreated condition=>P vs T. The same comparison was made for the irradiated batches with or without treatment=>example: PUVA vs UVA.

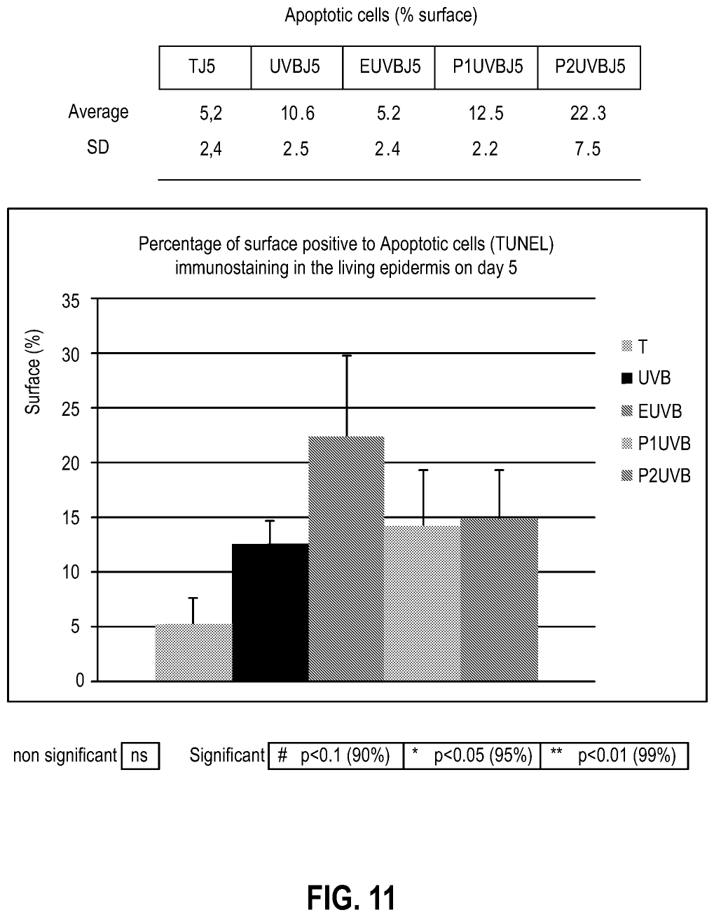

The percentage of surface occupied by apoptotic cells in the epidermis for all samples is shown in FIG. 11. On day 5, TJ5 apoptotic cells represent 5.2% of the surface of the epidermis. The UVB irradiations (UVBJ5 vs TJ5) induced a significant increase of 142% of apoptotic cells formation in the epidermis. Effect of product application on apoptotic cells formation was compared to UVBJ5. The excipient induced a significant increase of 78%, P1 induced a non-significant increase of 13%, and P2 induced a non-significant increase of 18%.

Effect of product application on apoptotic cells formation was compared to EUVBJ5. P1 induced a significant decrease of 36% and P2 induced a significant decrease of 34%.

In conclusion, the (SLT-008 0.1% solution) shows good DNA protection activity by reducing the levels of thymine dimers and apoptotic cell formation upon UVB irradiations (vs EUVBJ5), by 24% and 51% respectively. P2 (SLT-008 0.5% solution) also shows good DNA protection activity by reducing the levels of thymine dimers and apoptotic cell formation upon UVB irradiations (vs EUVBJ5), by 36% and 34% respectively.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound of the following Formula (I): 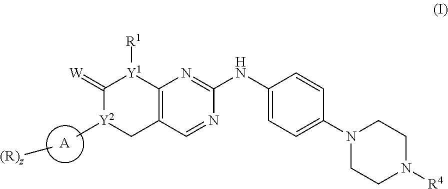
wherein W is S or O;
Y¹ is N or CR^{A};
Y² is N or CR^{B};
Ring A is mono- or multi-ring carbon alicyclic group, mono- or multi-ring or heteroalicyclic group, mono- or multi-ring carbocyclic aryl group or mono- or multi-ring carbocyclic aryl heteroaryl group;
each R is the same or different substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine;
z is an integer from 0 to a value permitted by the valence of Ring A;
R^{A} and R^{B} are independently H, halogen, —OH, —NH₂, —CN, or substituted or unsubstituted alky;
R¹ is is substituted or unsubstituted mono- or multi-ring carbon alicyclic group, substituted or unsubstituted mono- or multi-ring heteroalicyclic group, substituted or unsubstituted mono- or multi-ring carbocyclic aryl group or substituted or unsubstituted mono- or multi-ring heteroaryl group; and
R⁴ is H or substituted or unsubstituted alkyl;
and pharmaceutically acceptable salts thereof.

2. A compound of claim 1 wherein each R is the same or different substituted or unsubstituted alkyl or halogen.

3. A compound of claim 1 wherein each R is the same or different substituted or unsubstituted alkyl or chloro.

4. A compound of claim 1 wherein each R is the same or different substituted or unsubstituted alkyl.

5. A compound of claim 1 wherein Ring A is a mono- or multi-ring carbon alicyclic group or mono- or multi-ring heteroalicyclic group.

6. A compound of claim 1 wherein Ring A is a mono- or multi-ring carbocyclic aryl group or mono- or multi-ring heteroaryl group.

7. A compound of claim 1 wherein Ring A is a mono or multi-ring carbocyclic aryl group.

8. A compound of claim 1 wherein the compound is of the following Formula (II): 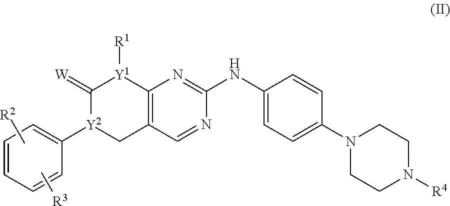
wherein W is S or O;
Y¹ is N or CR^{A};
Y² is N or CR^{B};
R^{A} and R^{B} are independently H, halogen, —OH, —NH₂, —CN, or substituted or unsubstituted alky;
R¹ is substituted or unsubstituted multi-ring carbon alicyclic or heteroalicyclic group;
R² and R³ are each independently H, substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine; and
R⁴ is H or substituted or unsubstituted alkyl; and pharmaceutically acceptable salts thereof.

9. A compound of claim 1 wherein the compound is of the following Formula (III): 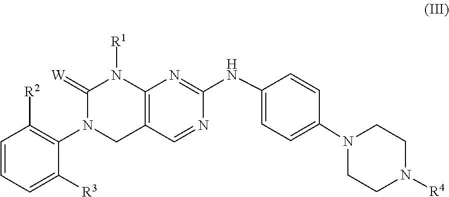
wherein in Formula (III):
W is S or O;
R¹ is a substituted or unsubstituted multi-ring carbon alicyclic or heteroalicyclic group;
R² and R³ are each independently are each independently H, substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine; and
R⁴ is H or substituted or unsubstituted alkyl;
and pharmaceutically acceptable salts thereof.

10. A compound of claim 1 wherein the compound is of the following Formula (IV): 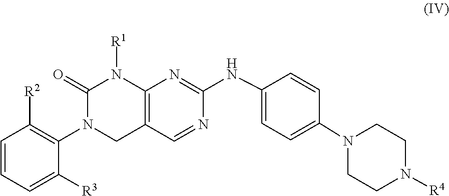
wherein in Formula (IV):
R¹ is a substituted or unsubstituted multi-ring carbon alicyclic or heteroalicyclic group;
R², and R³ are each independently are each independently H, substituted or unsubstituted alkyl, halogen, hydroxyl, cyano, amino, substituted or unsubstituted alkoxy, substituted or unsubstituted alkylthio, substituted or unsubstituted alkylsulfone, or substituted or unsubstituted alkylamine; and
R⁴ is H or substituted or unsubstituted alkyl; and pharmaceutically acceptable salts thereof.

11. A compound of claim 1 wherein R² and R³ are independently substituted or unsubstituted alkyl or halogen.

12. A compound of claim 1 wherein R² and R³ are independently substituted or unsubstituted alkyl or chloro.

13. A compound of claim 1 wherein R² and R³ are the same or different substituted or unsubstituted alkyl.

14. A compound of claim 1 wherein the compound is of the following Formula (V): 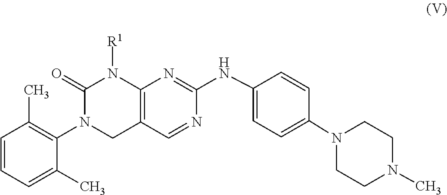
wherein in Formula (V):
R¹ is a substituted or unsubstituted multi-ring carbon alicyclic or heteroalicyclic group; and pharmaceutically acceptable salts thereof.

15. A compound of claim 1 wherein R¹ is an optionally substituted multi-ring carbon alicyclic group.

16. A compound of claim 1 wherein R¹ has a structure of the following Formula (VIa): 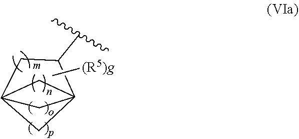
wherein in Formula (VIa):
m is an interger from 0 to 5;
n is an integer from 0 to 3;
o is an integer from 0 to 3;
p is an integer from 0 to 5,
each R⁵ is the same or different non-hydrogen substituent such as hydroxyl, halo, optionally substituted alkyl or optionally substituted heteroalkyl,
g is 0 or a positive integer, and
at least one of m, n, o, or p is a positive integer.

17. A compound of claim 1 wherein R¹ is an optionally substituted adamantyl, optionally substituted norbornyl, optionally substituted cyclo [2,2,2] octanyl or optionally substituted bicyclo[3,3,1] nonanyl.

18. A compound of claim 1 wherein R¹ is an optionally substituted multi-ring heteroalicyclic group.

19. A compound of claim 1 wherein R¹ is an optionally substituted thionorbonyl or optionally substituted oxonorbonyl.

20. A compound that is: 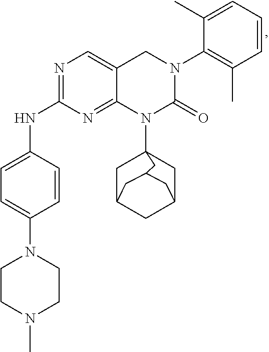 [SMILES: Cc1cccc(C)c1N1Cc2cnc(Nc3ccc(N4CCN(C)CC4)cc3)nc2N(C23CC4CC(CC(C4)C2)C3)C1=O]
or a pharmaceutically acceptable salt thereof.

21. A compound that is: 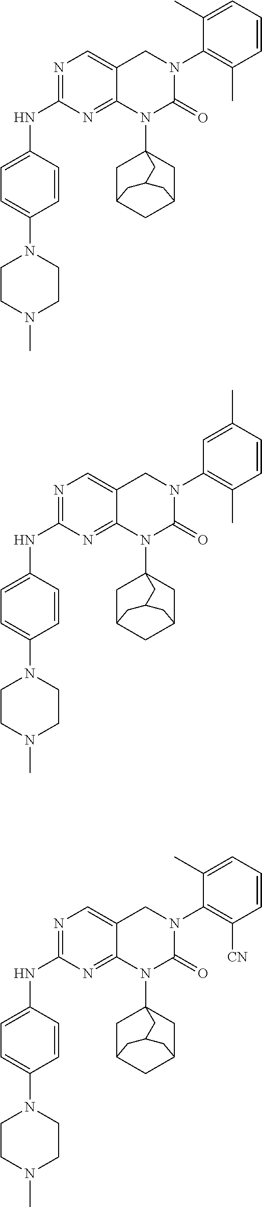 [SMILES: Cc1cccc(C)c1N1Cc2cnc(Nc3ccc(N4CCN(C)CC4)cc3)nc2N(C23CC4CC(CC(C4)C2)C3)C1=O ; Cc1ccc(C)c(N2Cc3cnc(Nc4ccc(N5CCN(C)CC5)cc4)nc3N(C34CC5CC(CC(C5)C3)C4)C2=O)c1 ; Cc1cccc(C#N)c1N1Cc2cnc(Nc3ccc(N4CCN(C)CC4)cc3)nc2N(C23CC4CC(CC(C4)C2)C3)C1=O] 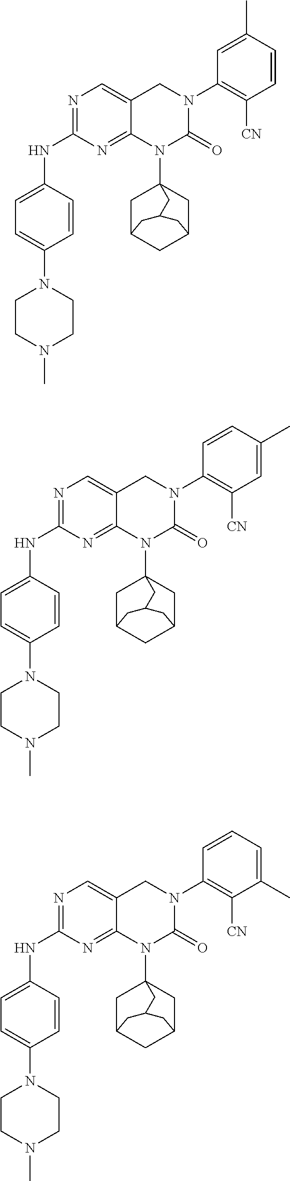 [SMILES: Cc1ccc(C#N)c(N2Cc3cnc(Nc4ccc(N5CCN(C)CC5)cc4)nc3N(C34CC5CC(CC(C5)C3)C4)C2=O)c1 ; Cc1ccc(N2Cc3cnc(Nc4ccc(N5CCN(C)CC5)cc4)nc3N(C34CC5CC(CC(C5)C3)C4)C2=O)c(C#N)c1 ; Cc1cccc(N2Cc3cnc(Nc4ccc(N5CCN(C)CC5)cc4)nc3N(C34CC5CC(CC(C5)C3)C4)C2=O)c1C#N] 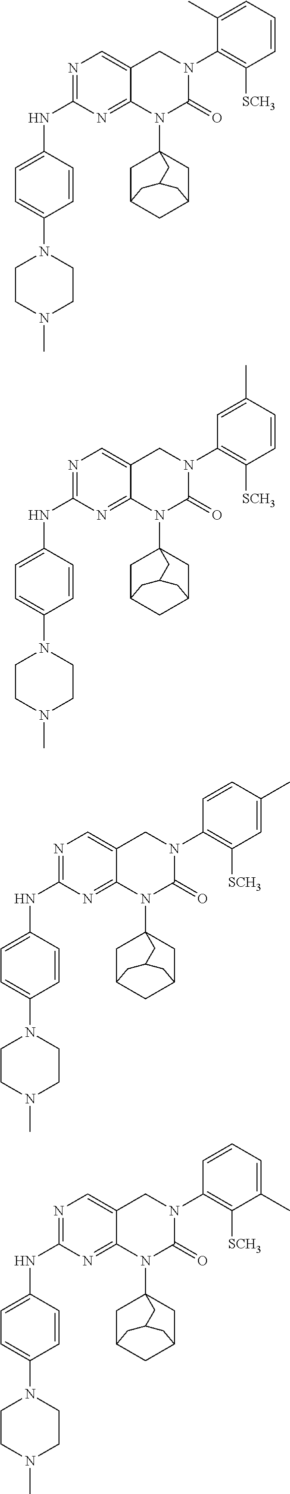 [SMILES: CSc1cccc(C)c1N1Cc2cnc(Nc3ccc(N4CCN(C)CC4)cc3)nc2N(C23CC4CC(CC(C4)C2)C3)C1=O ; CSc1ccc(C)cc1N1Cc2cnc(Nc3ccc(N4CCN(C)CC4)cc3)nc2N(C23CC4CC(CC(C4)C2)C3)C1=O ; CSc1cc(C)ccc1N1Cc2cnc(Nc3ccc(N4CCN(C)CC4)cc3)nc2N(C23CC4CC(CC(C4)C2)C3)C1=O ; CSc1c(C)cccc1N1Cc2cnc(Nc3ccc(N4CCN(C)CC4)cc3)nc2N(C23CC4CC(CC(C4)C2)C3)C1=O] 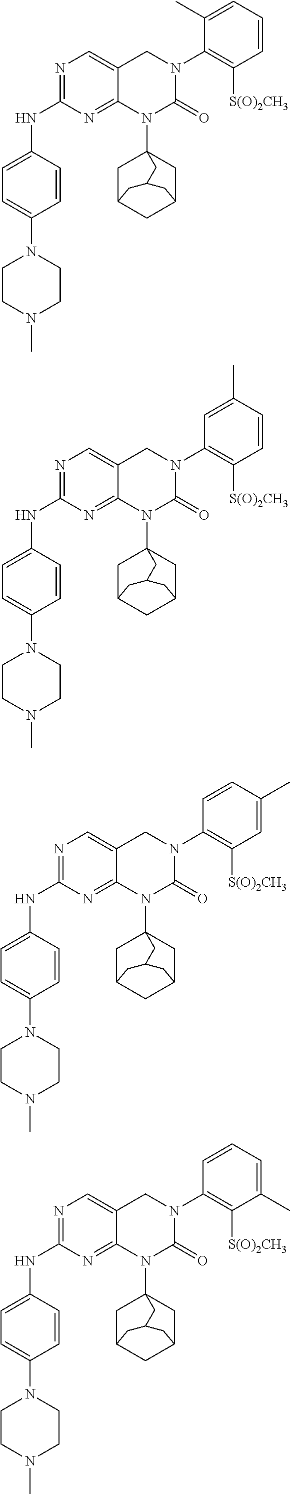 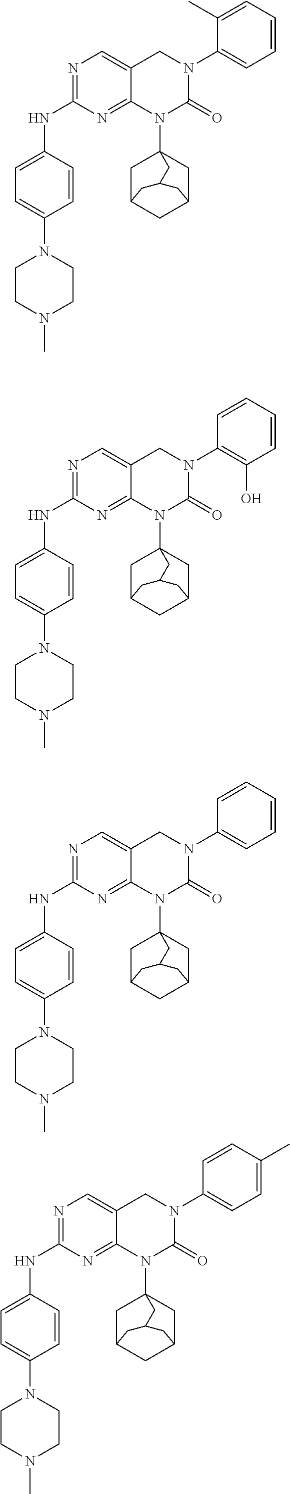 [SMILES: Cc1ccccc1N1Cc2cnc(Nc3ccc(N4CCN(C)CC4)cc3)nc2N(C23CC4CC(CC(C4)C2)C3)C1=O ; CN1CCN(c2ccc(Nc3ncc4c(n3)N(C35CC6CC(CC(C6)C3)C5)C(=O)N(c3ccccc3O)C4)cc2)CC1 ; CN1CCN(c2ccc(Nc3ncc4c(n3)N(C35CC6CC(CC(C6)C3)C5)C(=O)N(c3ccccc3)C4)cc2)CC1 ; Cc1ccc(N2Cc3cnc(Nc4ccc(N5CCN(C)CC5)cc4)nc3N(C34CC5CC(CC(C5)C3)C4)C2=O)cc1] 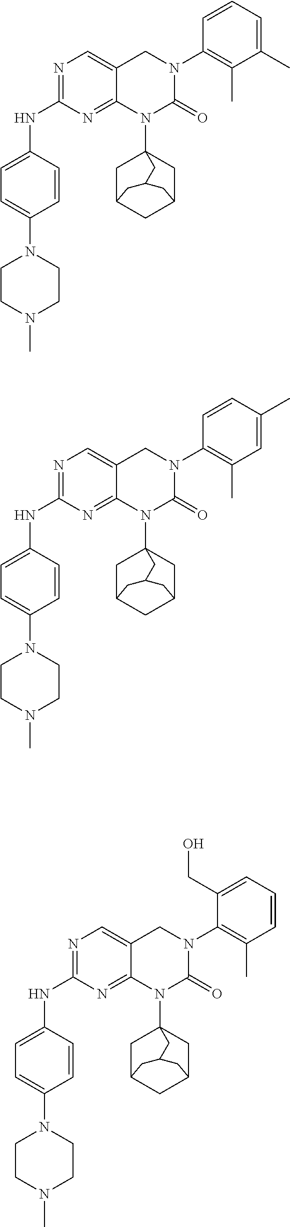 [SMILES: Cc1cccc(N2Cc3cnc(Nc4ccc(N5CCN(C)CC5)cc4)nc3N(C34CC5CC(CC(C5)C3)C4)C2=O)c1C ; Cc1ccc(N2Cc3cnc(Nc4ccc(N5CCN(C)CC5)cc4)nc3N(C34CC5CC(CC(C5)C3)C4)C2=O)c(C)c1 ; Cc1cccc(CO)c1N1Cc2cnc(Nc3ccc(N4CCN(C)CC4)cc3)nc2N(C23CC4CC(CC(C4)C2)C3)C1=O] 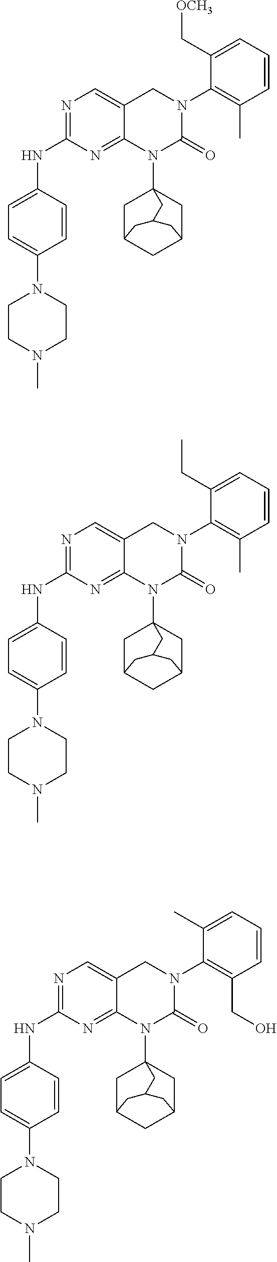 [SMILES: COCc1cccc(C)c1N1Cc2cnc(Nc3ccc(N4CCN(C)CC4)cc3)nc2N(C23CC4CC(CC(C4)C2)C3)C1=O ; CCc1cccc(C)c1N1Cc2cnc(Nc3ccc(N4CCN(C)CC4)cc3)nc2N(C23CC4CC(CC(C4)C2)C3)C1=O ; Cc1cccc(CO)c1N1Cc2cnc(Nc3ccc(N4CCN(C)CC4)cc3)nc2N(C23CC4CC(CC(C4)C2)C3)C1=O] 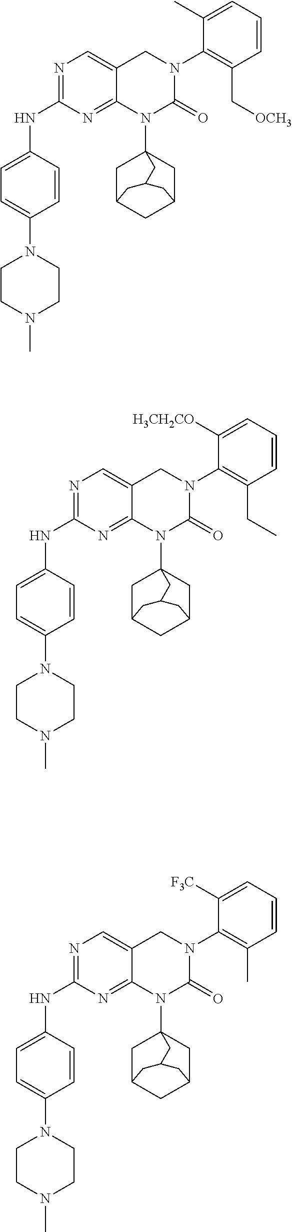 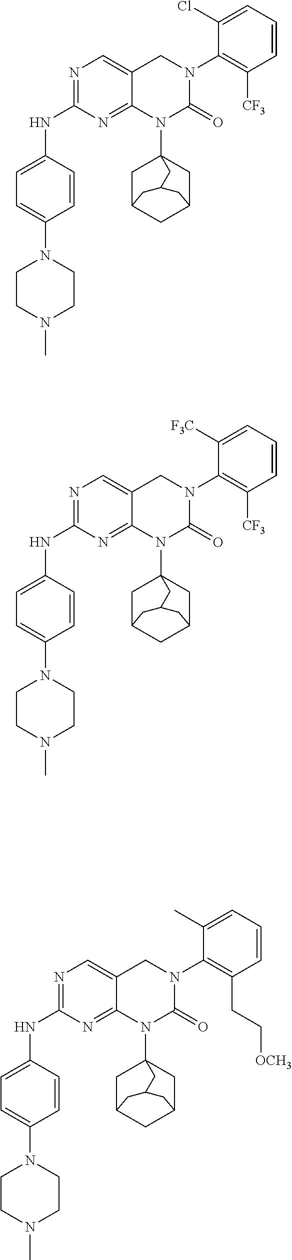 [SMILES: CN1CCN(c2ccc(Nc3ncc4c(n3)N(C35CC6CC(CC(C6)C3)C5)C(=O)N(c3c(Cl)cccc3C(F)(F)F)C4)cc2)CC1 ; CN1CCN(c2ccc(Nc3ncc4c(n3)N(C35CC6CC(CC(C6)C3)C5)C(=O)N(c3c(C(F)(F)F)cccc3C(F)(F)F)C4)cc2)CC1 ; COCCc1cccc(C)c1N1Cc2cnc(Nc3ccc(N4CCN(C)CC4)cc3)nc2N(C23CC4CC(CC(C4)C2)C3)C1=O] 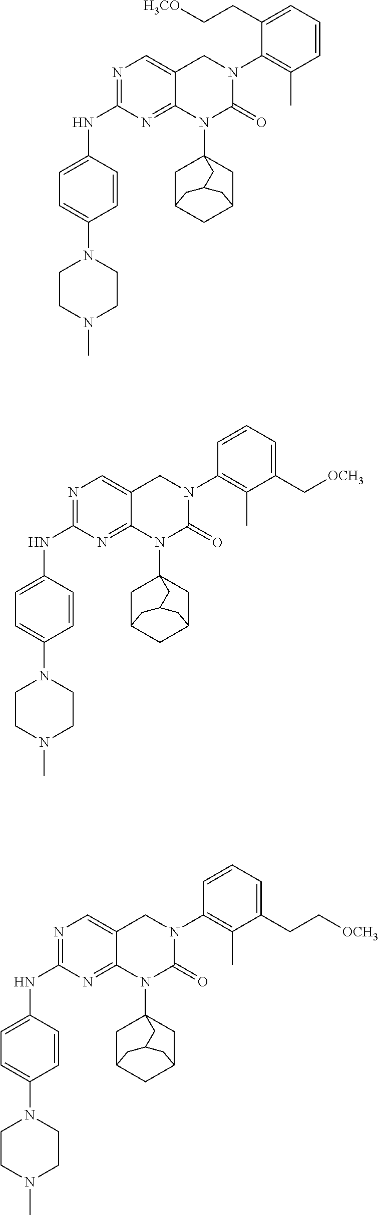 [SMILES: COCCc1cccc(C)c1N1Cc2cnc(Nc3ccc(N4CCN(C)CC4)cc3)nc2N(C23CC4CC(CC(C4)C2)C3)C1=O ; COCc1cccc(N2Cc3cnc(Nc4ccc(N5CCN(C)CC5)cc4)nc3N(C34CC5CC(CC(C5)C3)C4)C2=O)c1C ; COCCc1cccc(N2Cc3cnc(Nc4ccc(N5CCN(C)CC5)cc4)nc3N(C34CC5CC(CC(C5)C3)C4)C2=O)c1C] 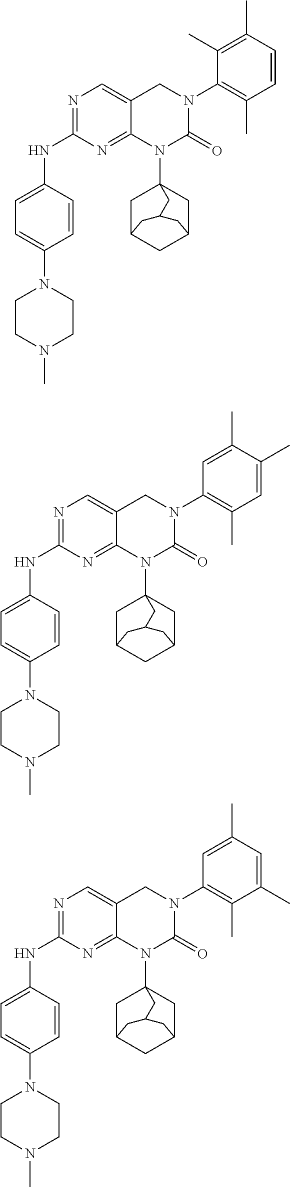 [SMILES: Cc1ccc(C)c(N2Cc3cnc(Nc4ccc(N5CCN(C)CC5)cc4)nc3N(C34CC5CC(CC(C5)C3)C4)C2=O)c1C ; Cc1cc(C)c(N2Cc3cnc(Nc4ccc(N5CCN(C)CC5)cc4)nc3N(C34CC5CC(CC(C5)C3)C4)C2=O)cc1C ; Cc1cc(C)c(C)c(N2Cc3cnc(Nc4ccc(N5CCN(C)CC5)cc4)nc3N(C34CC5CC(CC(C5)C3)C4)C2=O)c1] 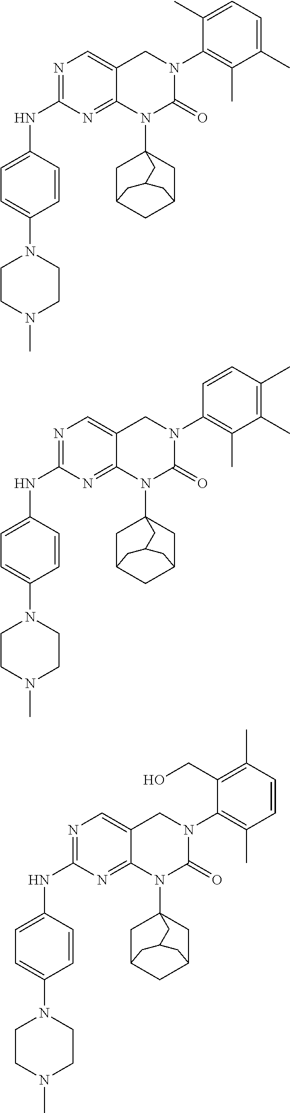 [SMILES: Cc1ccc(C)c(N2Cc3cnc(Nc4ccc(N5CCN(C)CC5)cc4)nc3N(C34CC5CC(CC(C5)C3)C4)C2=O)c1C ; Cc1ccc(N2Cc3cnc(Nc4ccc(N5CCN(C)CC5)cc4)nc3N(C34CC5CC(CC(C5)C3)C4)C2=O)c(C)c1C ; Cc1ccc(C)c(N2Cc3cnc(Nc4ccc(N5CCN(C)CC5)cc4)nc3N(C34CC5CC(CC(C5)C3)C4)C2=O)c1CO] 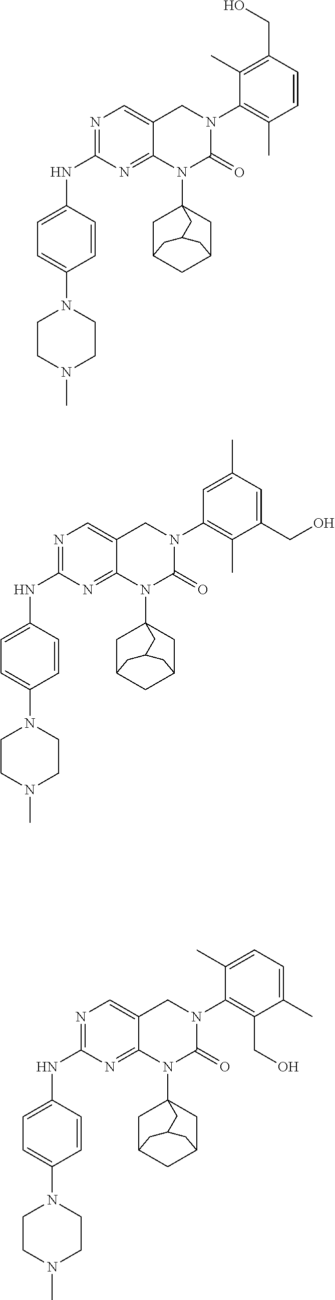 [SMILES: Cc1ccc(CO)c(C)c1N1Cc2cnc(Nc3ccc(N4CCN(C)CC4)cc3)nc2N(C23CC4CC(CC(C4)C2)C3)C1=O ; Cc1cc(CO)c(C)c(N2Cc3cnc(Nc4ccc(N5CCN(C)CC5)cc4)nc3N(C34CC5CC(CC(C5)C3)C4)C2=O)c1 ; Cc1ccc(C)c(N2Cc3cnc(Nc4ccc(N5CCN(C)CC5)cc4)nc3N(C34CC5CC(CC(C5)C3)C4)C2=O)c1CO] 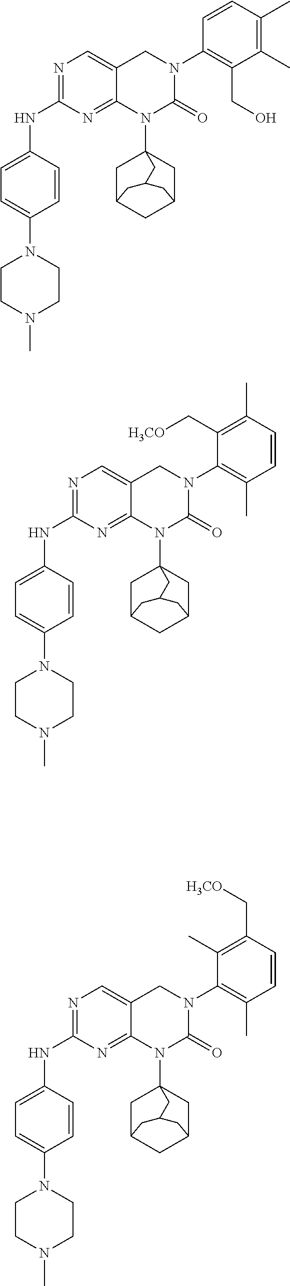 [SMILES: Cc1ccc(N2Cc3cnc(Nc4ccc(N5CCN(C)CC5)cc4)nc3N(C34CC5CC(CC(C5)C3)C4)C2=O)c(CO)c1C ; COCc1c(C)ccc(C)c1N1Cc2cnc(Nc3ccc(N4CCN(C)CC4)cc3)nc2N(C23CC4CC(CC(C4)C2)C3)C1=O ; COCc1ccc(C)c(N2Cc3cnc(Nc4ccc(N5CCN(C)CC5)cc4)nc3N(C34CC5CC(CC(C5)C3)C4)C2=O)c1C] 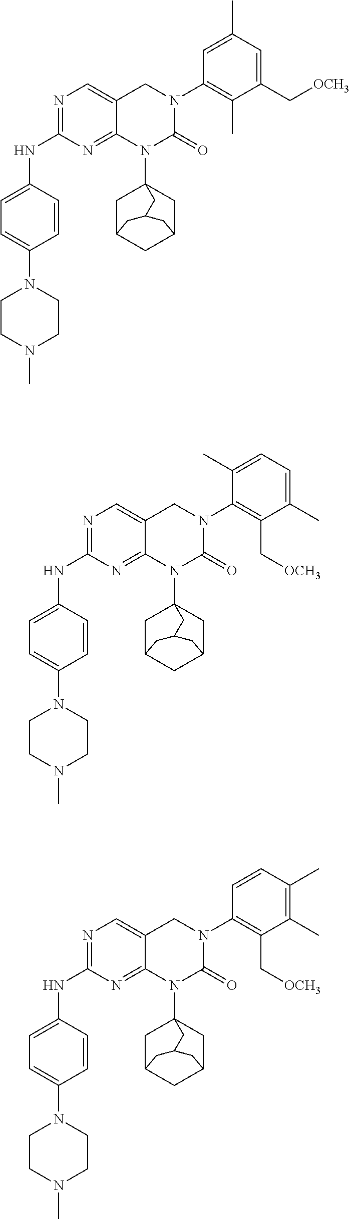 [SMILES: COCc1cc(C)cc(N2Cc3cnc(Nc4ccc(N5CCN(C)CC5)cc4)nc3N(C34CC5CC(CC(C5)C3)C4)C2=O)c1C ; COCc1c(C)ccc(C)c1N1Cc2cnc(Nc3ccc(N4CCN(C)CC4)cc3)nc2N(C23CC4CC(CC(C4)C2)C3)C1=O ; COCc1c(N2Cc3cnc(Nc4ccc(N5CCN(C)CC5)cc4)nc3N(C34CC5CC(CC(C5)C3)C4)C2=O)ccc(C)c1C] 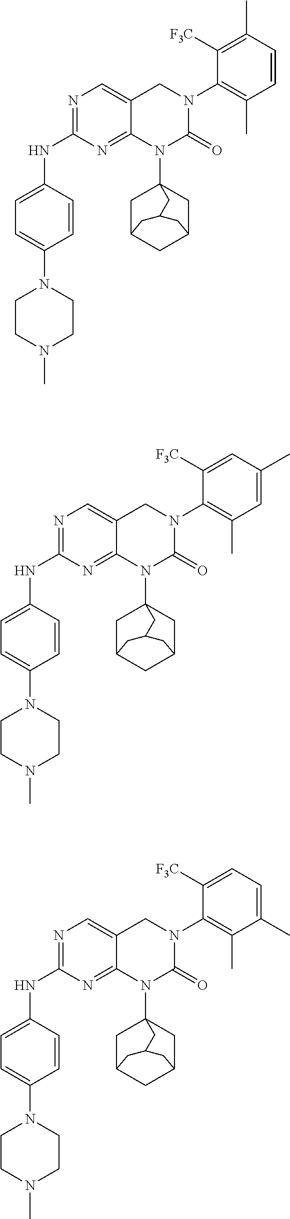 [SMILES: Cc1ccc(C)c(C(F)(F)F)c1N1Cc2cnc(Nc3ccc(N4CCN(C)CC4)cc3)nc2N(C23CC4CC(CC(C4)C2)C3)C1=O ; Cc1cc(C)c(N2Cc3cnc(Nc4ccc(N5CCN(C)CC5)cc4)nc3N(C34CC5CC(CC(C5)C3)C4)C2=O)c(C(F)(F)F)c1 ; Cc1ccc(C(F)(F)F)c(N2Cc3cnc(Nc4ccc(N5CCN(C)CC5)cc4)nc3N(C34CC5CC(CC(C5)C3)C4)C2=O)c1C] 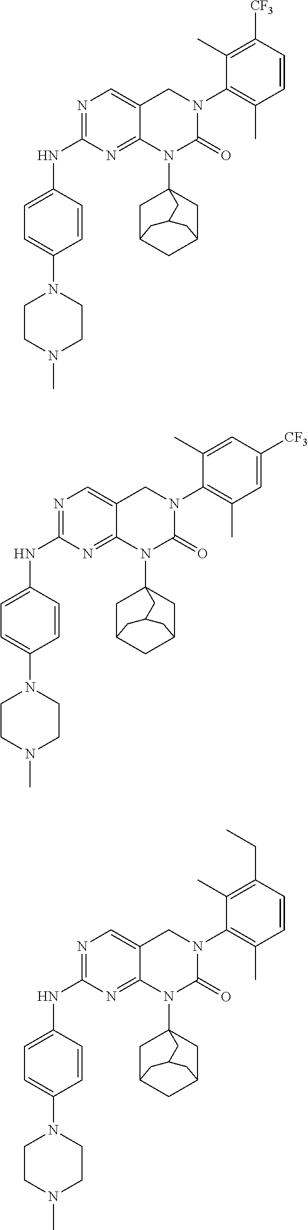 [SMILES: Cc1ccc(C(F)(F)F)c(C)c1N1Cc2cnc(Nc3ccc(N4CCN(C)CC4)cc3)nc2N(C23CC4CC(CC(C4)C2)C3)C1=O ; Cc1cc(C(F)(F)F)cc(C)c1N1Cc2cnc(Nc3ccc(N4CCN(C)CC4)cc3)nc2N(C23CC4CC(CC(C4)C2)C3)C1=O ; CCc1ccc(C)c(N2Cc3cnc(Nc4ccc(N5CCN(C)CC5)cc4)nc3N(C34CC5CC(CC(C5)C3)C4)C2=O)c1C] 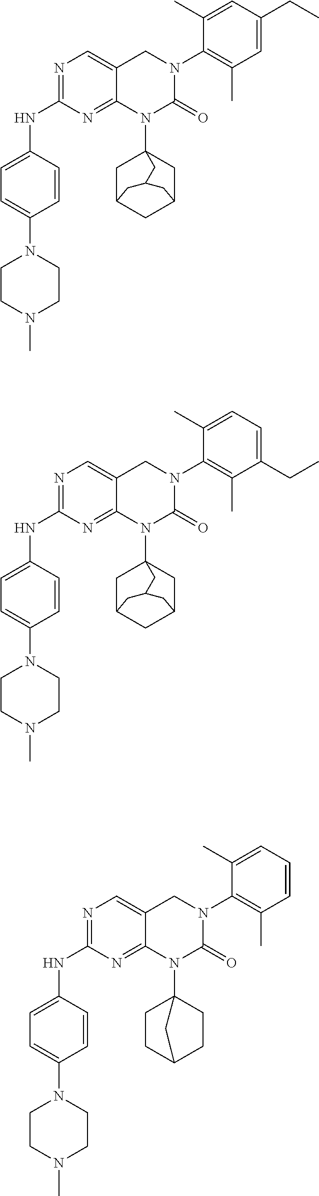 [SMILES: CCc1cc(C)c(N2Cc3cnc(Nc4ccc(N5CCN(C)CC5)cc4)nc3N(C34CC5CC(CC(C5)C3)C4)C2=O)c(C)c1 ; CCc1ccc(C)c(N2Cc3cnc(Nc4ccc(N5CCN(C)CC5)cc4)nc3N(C34CC5CC(CC(C5)C3)C4)C2=O)c1C ; Cc1cccc(C)c1N1Cc2cnc(Nc3ccc(N4CCN(C)CC4)cc3)nc2N(C23CCC(CC2)C3)C1=O] 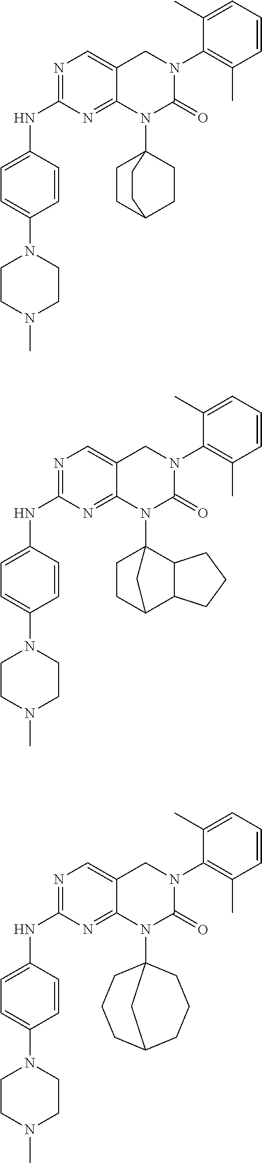 [SMILES: Cc1cccc(C)c1N1Cc2cnc(Nc3ccc(N4CCN(C)CC4)cc3)nc2N(C23CCC(CC2)CC3)C1=O ; Cc1cccc(C)c1N1Cc2cnc(Nc3ccc(N4CCN(C)CC4)cc3)nc2N(C23CCC(C2)C2CCCC23)C1=O ; Cc1cccc(C)c1N1Cc2cnc(Nc3ccc(N4CCN(C)CC4)cc3)nc2N(C23CCCC(CCC2)C3)C1=O] 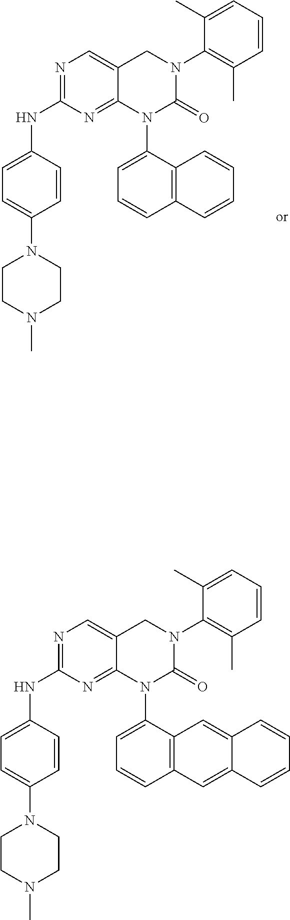 [SMILES: Cc1cccc(C)c1N1Cc2cnc(Nc3ccc(N4CCN(C)CC4)cc3)nc2N(c2cccc3ccccc23)C1=O ; Cc1cccc(C)c1N1Cc2cnc(Nc3ccc(N4CCN(C)CC4)cc3)nc2N(c2cccc3cc4ccccc4cc23)C1=O]
or a pharmaceutically acceptable salt thereof.

22. A pharmaceutical composition comprising a compound of claim 1 and one or more pharmaceutically acceptable carriers.

23. A method for treating a subject suffering from or susceptible to a skin-related disorder or disease, comprising
administering to the subject an effective amount of a compound or composition of claim 1.

24. The method of claim 23 wherein the subject is identified as suffering from a skin-related disorder or disease and the compound or composition in administered to the identified subject.

25. A method for treating a subject suffering from or susceptible to rosacea, comprising, administering to the subject an effective amount of a compound or composition of claim 1.

26-28. (canceled)

29. A method of increasing pigmentation in a tissue of a subject, said method comprising administering to the subject a compound or composition of claim 1, in an amount sufficient to increase melanin production, thereby increasing pigmentation in the tissue of the subject.

30. (canceled)

31. A method of increasing cellular DNA stability in the skin tissue of a subject in need thereof, comprising administering to the subject a compound or composition of claim 1, in an amount sufficient to decrease apoptosis and/or thymine dimer formation in the cellular DNA of the skin tissue, thereby increasing cellular DNA stability in the skin tissue of the subject.
